SEQUENCE LISTING (14 sequences)

SEQ ID NO: 1 - DNA, 21 bp, Mus musculus
cattgtgcagagaacccagaa

SEQ ID NO: 2 - DNA, 21 bp, Mus musculus
atgtcctccgcatacttgaac

SEQ ID NO: 3 - DNA, 23 bp, Mus musculus
ggcagttcagggaggaatcatgg

SEQ ID NO: 4 - DNA, 22 bp, Mus musculus
aaagcgccaggtggcatagctc

SEQ ID NO: 5 - DNA, 20 bp, Mus musculus
agcagtttcctcttcattcc

SEQ ID NO: 6 - DNA, 20 bp, Mus musculus
ccgagtcccagtgccagagt

SEQ ID NO: 7 - DNA, 20 bp, Mus musculus
cgtgggggagagtagtgtag

SEQ ID NO: 8 - DNA, 20 bp, Mus musculus
gggaggggagatagaggaga

SEQ ID NO: 9 - DNA, 19 bp, Mus musculus
agcccaagttctggagatg

SEQ ID NO: 10 - DNA, 20 bp, Mus musculus
ggtggttccattggcattgg

SEQ ID NO: 11 - DNA, 19 bp, Mus musculus
accatcaggccaggaggaa

SEQ ID NO: 12 - DNA, 20 bp, Mus musculus
acaaagcataccacaaacac

SEQ ID NO: 13 - DNA, 20 bp, Mus musculus
tgtattctttatttgctttg

SEQ ID NO: 14 - DNA, 20 bp, Mus musculus
cgtccctttggttttgttgt

(19) United States
(12) Patent Application Publication
(10) Pub. No.: US 2005/0053612 A1
(43) Pub. Date: Mar. 10, 2005

(54) Nucleotide regulation of immune responses

(76) Inventors: Richard D. Granstein, Scarsdale, NY (US); John A. Wagner, New York, NY (US)

Correspondence Address: Schwegman, Lundberg, Woessner & Kluth, P.A., P.O. Box 2938, Minneapolis, MN, 55402, US

(21) Appl. No.: 10/919,790

(22) Filed: Aug. 17, 2004

Related U.S. Application Data
(60) Provisional application No. 60/496,856, filed on Aug. 20, 2003.

(51) Int. Cl. C12P 21/04; A61K 39/38; A61K 39/00; A61K 31/522; A61K 31/675

(52) U.S. Cl. 424/184.1; 514/46; 514/47; 514/81; 514/263.31

(57) ABSTRACT

The invention provides purine receptor agonists and antagonists that are useful for modulating an immune response in an animal. Methods for modulating an inmmune response in an animal are also provided.

This application claims priority from U.S. Application Ser. No. 60/496,856 filed Aug. 20, 2003.

### GOVERNMENT FUNDING

The invention described in this application was made with funds from the National Institute of Health, Grant Numbers AR42429 and AI 59226. The United States government has certain rights in the invention.

### FIELD OF THE INVENTION

The invention relates to compositions and methods for modulating the immune response, particularly the use of purine receptor agonists and antagonists to regulate immunity.

### BACKGROUND OF THE INVENTION

Vaccines have proven to be successful, highly acceptable methods for the prevention of infectious disease. They are cost effective, and do not induce antibiotic resistance to the target pathogen or affect normal flora present in the host. In many cases, such as when inducing anti-viral immunity, vaccines can prevent a disease for which there are no viable curative or ameliorative treatments available.

Vaccines function by triggering the immune system to mount a response to an agent, or antigen, typically an infectious organism or a portion thereof that is introduced into the body in a non-infectious or non-pathogenic form. Once the immune system has been “primed” or sensitized to the organism, later exposure of the immune system to this organism as an infectious pathogen results in a rapid and robust immune response that destroys the pathogen before it can multiply and infect enough cells in the host organism to cause disease symptoms.

In many cases, it is necessary to enhance the immune response to the antigens present in a vaccine in order to stimulate the immune system to a sufficient extent to make a vaccine effective, i.e., to confer immunity. Many protein and most peptide and carbohydrate antigens, administered alone, do not elicit a sufficient antibody and/or cell mediated response to confer immunity. Such antigens need to be presented to the immune system in such a way that they will be recognized as foreign and will elicit an immune response. To this end, additives (adjuvants) have been devised that immobilize antigens and/or stimulate the immune response.

The best known adjuvant, Freund's complete adjuvant, consists of a mixture of mycobacteria in an oil/water emulsion. Freund's adjuvant works in two ways: first, it enhances cell and humoral-mediated immunity, and second, it blocks rapid dispersal of the antigen challenge (the “depot effect”). However, due to frequent toxic physiological and immunological reactions to this material, Freund's adjuvant cannot be used in humans.

Another molecule that has been shown to have immunostimulatory or adjuvant activity is endotoxin, also known as lipopolysaccharide (LPS). LPS stimulates the immune system by triggering an “innate” immune response—a response that has evolved to enable an organism to recognize endotoxin (and the invading bacteria of which it is a component) without the need for the organism to have been previously exposed. While LPS is too toxic to be a viable adjuvant, molecules that are structurally related to endotoxin, such as monophosphoryl lipid A (“MPL”) have been tested as adjuvants in clinical trials. Currently, however, the only FDA-approved adjuvant for use in humans is aluminum salts (Alum) which are used to “depot” antigens by precipitation of the antigens. Alum also stimulates the immune response to antigens.

Thus, a recognized need exists for compositions that can be co-administered with antigens in order to stimulate the immune system to generate a more robust antibody response to the antigen than would be seen if the antigen were administered alone or with Alum. Moreover, a need exists for agents that can reduce autoimmunity and inflammation.

### SUMMARY OF THE INVENTION

These and other needs are achieved by present invention, which is directed to compositions and methods for modulating the immune system.

In one embodiment, the invention provides a method of stimulating an immune response to an immunogen that includes administering a composition that has the immunogen and an effective amount of ATP or ATPγS. The composition can, for example, be administered intradermally, subcutaneously, intravascularly or topically.

In another embodiment, the invention provides adjuvant compositions including an effective amount of a purine receptor agonist. Purine receptors recognized by the agonist can be on antigen presenting cells. The purine receptor agonist can, for example, be administered intradermally, subcutaneously, intravascularly or topically.

The purine receptor agonist can be any available purine receptor agonist. Examples include ATP or ATPγS. Other examples include a, α, βmethylene-ATP (α, βmATP), β, γmethylene-ATP (β, 65 mATP), 2-methylthio-ATP (2mSATP), CTP, dATP, UTP, UTPγS, UDP, α,βmethylene-adenosine 5′-triphosphate (α, βmATP), D-β, γmethylene-adenosine 5′-triphosphate (D-β, γmATP), 2-methylthio-adenosine 5′-triphosphate (2mSATP), 2-methylthioadenosine 5′-triphosphate, 2′,3′-O-(4-benzoylbenzoyl)adenosine 5′-triphosphate, hexythioadenosine 5-monophosphate, 2-[2-(4-aminophenyl)ethylthio]adenosine 5′-triphosphate, diadenosine tetraphosphate, diadenosine pentaphosphate, adenosine 5′-O-(2-thiodiphosphate), 2-methylthioadenosine 5′-diphosphate, 2-propylthio- D-β,γ-dichloromethylene-ATP or uridine 5′-O-(2-thiodiphosphate).

In some embodiments, the purine receptor agonist is a compound of formula I or II:

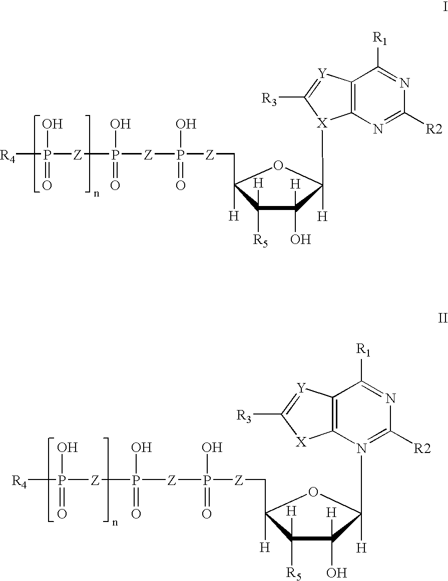

wherein:
R₁ and R₂, independently, are halogen or —R₆—(R₇)ₚ—R₈;
R₃ is H, halogen or —R₆—(R₇)ₚ—R₈, wherein p is 0 or 1;
R₄ is OH or SH;
R⁵ is OH or acetamido;
R₆ is NH or S;
R₇ is H or alkylene having from 1 to 10 carbon atoms;
R₈ is H, NH₂, CN, cycloalkyl having 3 to about 10 carbon atoms, or
aryl having 3 to about 20 carbon atoms;
X and Y are independently N or CH;
Z is O, S or CH₂; and
n is 0 or 1.

The invention also provides a composition for treating an undesirable immune response that includes a carrier and an effective amount of a purine receptor antagonist. The purine receptor antagonist can be administered to inhibit the function of a purine receptor.

Examples of purine receptor antagonists include oxidized ATP, pyridoxal-5′-phosphate-6-(2′-naphthylazo-6′-nitro-4′,8′-disulfonate), pyridoxal-5′-phosphate-6-azophenyl-2′,5′-disulfonic acid, pyridoxal-5′-phosphate-6-azophenyl-2′,4′-disulfonic acid, pyridoxal-5′-phosphate-6-azophenyl-4′-carboxylate, diinosine pentaphosphate, 8,8′-(carbonylbis(imino-3,1-phenylene carbonylimino)bis(1,3,5-naphthalenetrisulfonic acid), 8,8′-(carbonylbis(imino-4,1-phenylene carbonylimino-4,1-phenylene carbonylimino)bis(1,3,5-naphthalenetrisulfonic acid), Suramin, 2′,3′-O-(2,4,6-trinitrophenyl) adenosine triphosphate, reactive blue 2, brilliant blue G, 1-[N, O-bis(5-isoquinolonesulfonyl)-N-methyl-L-tyrosyl]-4-phenylpiperazine, hexamethylene amioride, oxidized ATP, adenosine 3′-phosphate 5′-phosphosulfate, 2′-deoxy-N⁶-methyladenosine-3′,5′-bisphosphate, (N)-methanocarba-N⁶-methyl-2-chloro-2′-deoxyadenosine-3′,5′-bisphosphate, 2-propylthio-D-β, γ-dichloromethylene-ATP, N⁶-[2-(methylthio)-ethyl]-2-(3,3,3-trifluoropropyl)thio-5′-adenylic acid, or N¹-(6-ethoxy-1,3-benzothiazol-2-yl-2(7-ethoxy-4-hydroxy-2,2-dioxo-2H-2-⁶benzo[4,5][1,3] thiazolo[2,3-c][1,2,4]thiadiazin-3-yl)-2oxo-1-ethanesulfonamide, 2-methylthioadenosine 5′-monophosphate.

The undesirable immune response can be, for example, an autoimmune disease, or a negative reaction against a therapeutic agent. Examples of autoimmune diseases that may be treated according to the invention include diabetes mellitus, arthritis, rheumatoid arthritis, juvenile rheumatoid arthritis, osteoarthritis, psoriatic arthritis, multiple sclerosis, myasthenia gravis, systemic lupus erythematosis, autoimmune thyroiditis, dermatitis, atopic dermatitis, eczematous dermatitis, psoriasis, Sjogren's Syndrome, keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves ophthalmopathy, sarcoidosis, primary biliary cirrhosis, uveitis posterior, or interstitial lung fibrosis. Examples of undesired immune responses against therapeutic agents include negative reactions against drugs, chemical agents, therapeutic antibodies (for example, against anti-CD 22 antibodies, antibodies against growth factors, erythropoietin or hybrid proteins such as alefacept) and the like.

The purine receptor antagonist is administered intradermally, subcutaneously, intramuscularly, intravenously or topically. In other embodiments, the purine receptor antagonist is administered at a site of inflammation.

The invention also provides methods for stimulating an immune response in an animal that include administering to the animal an antigen and an adjuvant composition having an effective amount of a purine receptor agonist. Any antigen contemplated by one of skill in the art can be administered with the adjuvant compositions of the invention.

The invention further provides methods for treating or preventing an undesirable immune response in an animal that include administering to the animal an effective amount of a purine receptor antagonist.

### BRIEF DESCRIPTION OF THE DRAWINGS

FIG. 1 graphically illustrates that intradermal administration of ATPγS at the site of induction of contact hypersensitivity leads to an exaggerated contact hypersensitivity response as measured at 24 hours. As illustrated, animals treated with both dinitrofluorobenzene (DNFB) and ATPγYS (group A) exhibited the greatest ear swelling. Animals treated with dinitrofluorobenzene (DNFB) alone (group B) exhibited intermediate levels of ear swelling. Animals treated with only ATPγS (group C) exhibited the least ear swelling. While animals treated with ATPγS alone (group C) apparently had less ear swelling than control animals who received PBS only (group D), the difference was not statistically significant. p=0.026 for A vs. B at 24 hours, not significant (NS) for C vs. D. (Student's two-tailed t-test for unpaired values. N=5 for all groups.)

FIG. 2 graphically illustrates that intradermal administration of ATPγS at the site of induction of contact hypersensitivity leads to an exaggerated contact hypersensitivity response as measured at 48 hours As illustrated, animals treated with both dinitrofluorobenzene (DNFB) and ATPγS (group A) exhibited the greatest ear swelling. Animals treated with dinitrofluorobenzene (DNFB) alone (group B) exhibited intermediate levels of ear swelling. Animals treated with only ATPγS (group C) exhibited the least ear swelling. While animals treated with ATPγS alone (group C) apparently had less ear swelling than control animals who received PBS only (group D), the difference was not statistically significant. p<0.004 for A vs. B at 48 hours, NS for C vs. D. (Student's two-tailed t-test for unpaired values. N=5 for all groups.)

FIG. 3 further illustrates that intradermal ATPγS augments the induction of contact hypersensitivity (CHS) near the site of administration. BALB/c mice received an intradermal injection ATPγS or PBS alone on the lower back. Fifteen minutes later, mice were sensitized by application of DNFB to the same area or at a distant site. Seven days later all mice were challenged by DNFB application to the ears. Ear swelling was measured at 24 hours. Mice initially sensitized to DNFB showed significant swelling upon DNFB challenge compared to mice not sensitized to DNFB. Mice injected with ATPγS prior to DNFB application at the injected site had an increased response upon challenge compared to mice injected with PBS alone at the site of injection (*p<0.05). Injection of ATPγS at a site distant from that of immunization had no effect on the response. Mean values±SD; N=5 per group. This experiment is representative of 3 experiments.

FIG. 4A-F illustrates that mice immunized with killed group A streptococcus (GAS) in the presence of ATPγS developed much smaller lesions than mice immunized with killed GAS alone. Groups of three C57BL/6 mice were injected intradermally with 10⁸ killed (freeze-thawed×4) GAS in 100 μl of medium containing 700 nanomoles of ATPγS or GAS in medium alone (without bacteria). Immunizations were repeated twice at 7 day intervals. One week after the last immunization all mice were shaved, hair was removed by chemical depilation with topical sodium thioglycollate and the mice were injected subcutaneously with 50 μl of mid-logarithmic growth phase (A600=0.6, about 5×107 c.f.u.) GAS complexed to Cytodex beads as a carrier. Lesions were measured daily. At day five the mean areas of the lesions were 25.01±10.35 mm² for mice immunized without ATPγS (FIG. 4D-F, bottom three panels) versus 2.80±1.11 mm² for mice immunized with ATPγS (FIG. 4A-C, top three panels) (p=0.021, student's two-tailed t-test for unpaired values).

FIG. 5 illustrates that mice immunized with tumor-associated antigens (TAA) and ATPγS had an enhanced delayed-type hypersensitivity (DTH) response compared to mice immunized with TAA alone. CAF₁ mice received an intradermal injection with TAA, TAA mixed with 30 nanomoles of ATPγS or PBS alone. One week later each mouse was challenged by footpad injection with TAA and footpad swelling measured at 24 and 48 hr. Mice immunized with TAA and ATPγS exhibited a significantly greater DTH response compared to mice immunized with TAA alone (p=0.013). Mean values±SD; N=5 per group. This experiment is representative of two experiments.

FIG. 6 illustrates that treatment of Langerhans cells (LCs) with ATPγS enhanced the antigen presenting ability of these cells as detected by interferon-γ (IFN-γ) production. LCs were cultured with the antigen KLH for 2 hours and then ATPγS was added for an additional 2 hours. The cells were then washed and co-cultured with the KLH-responsive TH1 clone HDK-1. Supernatants were collected after 72 hours and analyzed for IFN-γ production by ELISA. ATPγS augmented the ability of LCs to present KLH to HDK-1 cells in a dose-dependent manner. Exposure to ATPγS did not significantly change the background response seen in the negative control group (*p<0.05). Mean values±SD. This experiment was representative of 5 experiments.

FIG. 7A-I shows that ATPγS enhances expression of I-A, CD80 and CD86. XS106 cells were cultured in LPS and GM-CSF (FIG. 7D-F), or in LPS and GM-CSF with (FIG. 7G-I) or without (FIG. 7A-C) the addition of ATPγS. Cells were harvested at 48 hours and analyzed by FACS for expression of I-A^{k} (FIG. 7A, D and G), CD86 (FIG. 7B, E and H) and CD80 (FIG. 7C, F and I). This experiment was representative of 10 experiments. FIG. 7J illustrates that culturing XS106A cells in oxidized ATP inhibits the effect of ATPγS on CD80 expression, as assessed by cytofluorography.

FIG. 8A-C shows that ATPγS enhanced stimulated production of IL-1β and IL-12 while inhibiting IL-10. The XS106 cell line (with characteristics like that of Langerhans cells) was treated with increasing concentrations of ATPγS in the presence of GM-CSF/LPS (FIG. 8A and 8C) or GM-CSF alone (FIG. 8B). Supernatants were collected after 24 h hours and analyzed for cytokine production using ELISA. Stimulation with ATPγS produced a significant increase in IL-1β and IL-12 (FIG. 8A and 8B), and a decrease in IL-10 (FIG. 8C). Experiments were performed in triplicate (*p<0.05, **<0.01). Mean values±SD. This experiment is representative of 2 experiments for IL-12, 8 for IL-Iβ and 10 for IL-10.

FIG. 8D graphically illustrates that culturing XS106A cells in ATP also augmented secretion of IL-1β. The values on the x-axis indicate the molarity of te ATP. p=0.001 for 0.0 M vs. 10⁻³ M, p=0.005 for 0.0 M vs. 10⁻⁴ M, NS for 0.0 nM vs. 10⁻⁵ M and 0.0 nM vs. 10⁻⁶ M. (Student's two-tailed t-test for unpaired values.)

FIG. 9A-B shows that XS106 cells express mRNA for P2X1, P2X7, P2Y1, P2Y2, P2Y4, P2Y9 and P2Y11 receptors. Fragments of the expected sizes for the various P2X and P2Y receptors were obtained by reverse transcription-polymerase chain reaction (RT-PCR) assays. Reactions were run without reverse transcriptase to control for the possible presence of contaminating DNA.

### DETAILED DESCRIPTION OF THE INVENTION

The present invention is directed to the therapeutic agents useful for modulating the immune response. In one embodiment, the invention is directed to agents and methods for stimulating a cutaneous immune response. According to the invention, purine receptor agonists such as purine nucleoside triphosphates and analogs thereof can stimulate the cutaneous immune response.

Moreover, another aspect of the invention is directed to agents and methods useful for treating and preventing autoimmune and other inflammatory conditions. As provided herein purine receptor antagonists can be used to decrease the immune response, particularly the cutaneous immune response.

Hence, in one embodiment, the invention is directed to compositions and methods for increasing an immune response in an animal that includes administration of a purine receptor agonist and a substance against which it is desired to raise an immune response. A substance against which it is desired to raise an immune response is often referred to herein as an antigen, but it will be appreciated that in many circumstances the substance is not immunogenic in the subject animal without the intervention of an purine receptor agonist of the invention (or other adjuvant) and in fact may be entirely homologous to a molecule within the animal. A substance that is not normally antigenic within the subject to be immunized induces no detectable titer of antibody or cytotoxic T lymphocyte (CTL activity) against the substance after immunization of a person or an animal. This is often the case with many tumor antigens.

The agent, or antigen, used to prime the immune system can be the entire organism in a less infectious state, for example, an attenuated bacteria or virus, or in other cases, components of the organism such as carbohydrates, proteins or peptides representing various structural components of the organism. Antigens for use herein include any small molecule, peptide, polypeptide, polysaccharide, lipid, or combination thereof. Examples include toxins, viral polypeptides, bacterial proteins, tumor antigens, hormones and synthetic low molecular weight polypeptides (e.g., about from 3 to 15 residues). Hormones that can act as antigens in the invention include polypeptide reproductive hormones such as LHRH, inhibin or the prosequence of the alpha chain of inhibin, growth factors such as TGF-β or TGF-α, somatostatin, cell surface receptors for growth hormones, as well as chemical entities including drugs or haptens that may induce an immune response.

In other situations, one of skill in the art may wish to depress an inflammation or immune response in an animal. In these situations, a purine receptor antagonist can be administered to the animal. Situations where an inflammation or immune response may be diminished include undesired immune responses such as autoimmune conditions, tissue rejection and similar conditions that are described in more detail herein. Other unwanted immune responses that could be reduced or avoided by using the receptor antagonists and methods of the invention include negative responses against therapeutic agents such as insulin allergies and negative reactions against other peptide drugs.

The animals to be treated in accordance with this invention are those that are capable of mounting an immune response to an antigen. This includes mammals, particularly humans, but also commercial livestock such as beef, swine, goats, sheep, chickens, turkeys, ducks, geese and the like, as well as experimental animals such as mice, rats, rabbits, goats and the like.

Purine Receptor Agonists and Antagonists

According to the invention, ATP, ATP analogs and other agonists of purine receptors act as immunostimulants that can stimulate an immune response. In another aspect of the invention, agents that can antagonize the activity of purine receptors can be used to inhibit an undesirable immune response, for example, an autoimmune response or an immune reaction against a therapeutic agent.

ATP exerts different actions in various tissues and organs. These actions are mediated by distinct cell surface receptors, termed P2-purinoceptors. These P2 receptors are different from the adenosine receptors that are termed P1-purinoceptors. The distinction between different receptors is important because adenosine is a breakdown product of ATP. The P2-purinoceptors comprise two major families, P2X and P2Y. Each family consists of at least seven members (X₁₋₇ and Y₁₋₇). The P2X family represents cell membrane ligand-binding ion channels permeable to Na⁺, K⁺ and Ca⁺⁺. The P2Y-purinoceptors constitute G-protein-linked receptors, often coupled to phospholipase C and, hence, to inositol triphosphate formation. There are at least seven different subclasses of P2Y receptor, based upon agonist potency profiles. For a description of the various P2Y subtypes, see Abbrachio and Bumstock, Pharmac. Ther. 64, 445-475, 1994, the entire disclosure of which is incorporated herein by reference.

The existence of cellular receptors for purine nucleotides and nucleosides has been known for over 20 years. Receptors for nucleotides are known as P2 receptors. Twelve P2 receptors have been cloned and characterized. Di Virgilio et al., Blood 97:587-600, 2001. P2 receptors are divided into two families, the P2Y family of G protein-coupled receptors and the P2X family of ligand-gated ion channels.

According to the invention, any agonist of a P2 receptor can be used as an adjuvant to stimulate the immune system. Such agonists can bind to P2 receptors and stimulate the P2 receptor to perform its normal function. In some embodiments, ATP is used as the P2 receptor agonist. In other embodiments, ATP analogs are used as the P2 receptor agonist. Any agonist that can stimulate the P2 receptor to perform its normal function can be used as an adjuvant in the practice of the invention. In general, there is no need for hydrolysis of ATP to ADP in order to stimulate the immune system. But in some cases ADP or ADP analogs can be potent agonists at specific receptors.

In other embodiments, purine receptor antagonists can be used to diminish or decrease an undesirable immune response.

In some embodiments, the invention provides ATP analogs having formula I or II that can modulate the cutaneous immune response:

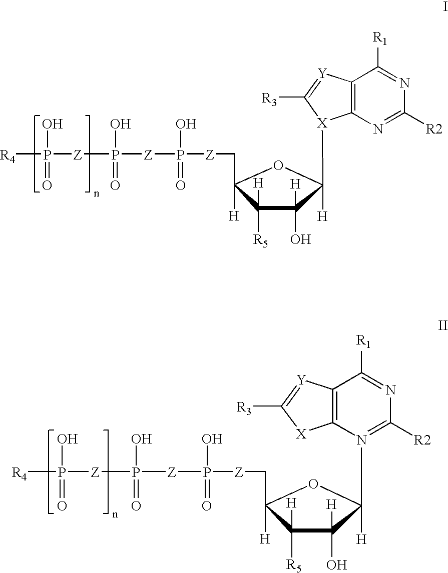

wherein:
R₁ and R₂, independently, are halogen or —R₆—(R₇)ₚ—R₈;
R₃ is H, halogen or —R₆—(R₇)ₚ—R₈, wherein p is 0 or 1;
R₄ is OH or SH;
R₅ is OH or acetamido;
R₆ is NH or S;
R₇ is H or alkylene having from 1 to 10 carbon atoms;
R₈ is H, NH₂, CN, cycloalkyl having 3 to about 10 carbon atoms, or aryl having 3 to about 20 carbon atoms;
X and Y are independently N or CH;
Z is O, S or CH₂; and
n is 0 or 1.
   In certain preferred embodiments, R₈ is —C₆H₁₁, —C₅H₉, —C₆H₅, —C₆H₄—NO₂, or —CH[C₆H₄(CH₃)] [C₆H₃(OCH₃)₂]. In other preferred embodiments R₃ is H, and R₁ and R₂ are NH₂, S or Cl, and in further preferred embodiments X and Y are N or CH.

The following general definitions are used, unless otherwise described: halo is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Aryloxy means aryl-O—.

More specifically, lower alkyl means (C₁-C₆) alkyl. Such (C₁-C₆) alkyl can be methyl, ethyl, propyl, isopropyl, butyl, iso-butyl, sec-butyl, pentyl, 3-pentyl, or hexyl. Lower alkoxy generally means (C₁-C₆) alkoxy; such (C₁-C₆) alkoxy can, for example, be methoxy, ethoxy, propoxy, isopropoxy, butoxy, iso-butoxy, sec-butoxy, pentoxy, 3-pentoxy, or hexyloxy. Preferred lower alkyl groups are (C₁-C₃) alkyl including methyl ethyl, propyl, isopropyl and the like. More preferred lower alkyl groups are methyl. Cycloalkyl groups generally have about 3 to about 10 carbon atoms, however some cycloalkyl groups have about 5 to about 6 carbon atoms. Lower acyl refers to a carbonyl group attached to a lower alkyl group (e.g., —CO—CH₃). Lower hydroxy alkyl refers to a hydroxy group attached to a lower alkyl or lower alkylene group (e.g. —CH₂—CH₂—OH).

The invention also provides compositions comprising the compound of formula I or II and a pharmaceutically acceptable carrier, preferably in an amount effective to modulate an immune response. For example, the following ATP analogs can be used in the compositions and during the practice of the invention to modulate immune responses: ATPγS, CTP, dATP, UTP, UTPγS, UDP, α, β methylene-adenosine 5′-triphosphate (α, βmATP), D-β, γmethylene-adenosine 5′-triphosphate (D-β, γmATP), 2-methylthio-adenosine 5′-triphosphate (2mSATP), 2-methylthioadenosine 5′-triphosphate, 2′,3′-O-(4-benzoylbenzoyl)adenosine 5′-triphosphate, hexythioadenosine 5-monophosphate, 2-[2-(4-aminophenyl)ethylthio]adenosine 5′-triphosphate, diadenosine tetraphosphate, diadenosine pentaphosphate, adenosine 5′-O-(2-thiodiphosphate), 2-methylthioadenosine 5′-diphosphate, 2-propylthio- D-β, γ-dichloromethylene-ATP or uridine 5′-O-(2-thiodiphosphate). Most of the foregoing ATP analogs can act as purine receptor agonists.

Analogs of ATP that can competitively bind to P2-purinoceptors and inhibit the normal function of P2 receptors can be used to depress an immune response or an inflammatory reaction in a mammal. Examples of such antagonists include pyridoxal-5′-phosphate-6-(2′-naphthylazo-6′-nitro-4′,8′-disulfonate), pyridoxal-5′-phosphate-6-azophenyl-2′,5′-disulfonic acid, pyridoxal-5′-phosphate-6-azophenyl-2′,4′-disulfonic acid, pyridoxal-5′-phosphate-6-azophenyl-4′-carboxylate, diinosine pentaphosphate, 8,8′ -(carbonylbis(imino-3,1-phenylene carbonylimino)bis( 1,3,5-naphthalenetrisulfonic acid), 8,8′-(carbonylbis(imino-4,1-phenylene carbonylimino-4,1-phenylene carbonylimino)bis(1,3,5-naphthalenetrisulfonic acid), Suramin, 2′,3′-O-(2,4,6-trinitrophenyl) adenosine triphosphate, reactive blue 2, brilliant blue G, 1-[N, O-bis(5-isoquinolonesulfonyl)-N-methyl-L-tyrosyl]-4-phenylpiperazine, hexamethylene amioride, oxidized ATP, adenosine 3′-phosphate 5′-phosphosulfate, 2′-deoxy-N⁶-methyladenosine-3′,5′-bisphosphate, (N)-methanocarba- N⁶-methyl-2-chloro-2′-deoxyadenosine-3′,5′-bisphosphate, 2-propylthio-D-β, γ-dichloromethylene-ATP, N⁶-[2-(methylthio)-ethyl]-2-(3,3,3-trifluoropropyl)thio-5′-adenylic acid, or N¹-(6-ethoxy-1,3-benzothiazol-2-yl-2(7-ethoxy-4-hydroxy-2,2-dioxo-2H-2-⁶benzo[4,5][1,3] thiazolo[2,3-c][1,2,4]thiadiazin-3-yl)-2oxo-1-ethanesulfonamide, 2-methylthioadenosine 5′-monophosphate.

Methods of Use

The purine receptor agonists and antagonists of the invention can be administered in an effective amount to modulate the immune system.

In one embodiment, a purine receptor agonist is used to boost an immune response. In other words, the purine receptor agonist can act as an adjuvant. An adjuvant effective amount of the agonists of the invention can be determined by the clinician taking into account the method and site of delivery of the agonists of the invention, whether the agonists of the invention are administered together with antigen, the inherent immunogenicity of the antigen, the desired form of the response (elevation of titer, prolongation of the response, or both), the presence of carriers and other considerations that will be apparent to those skilled in the art.

Doses of antigen will vary widely, and must be determined empirically. Where attenuated bacterial or tumor cells are serving as the target, generally about from 1×10⁶ to 50×10⁶ cells are administered per kilogram of body weight per dose. Each dose can be administered at a plurality of sites, and preferably by intradermal, subcutaneous or intramuscular injection. Generally the antigen will be administered together with an adjuvant of the invention although boosters with antigen may not require adjuvant. However, one must use a dose of agonist or antagonist that is less than the maximum tolerated dose (MTD). This will vary among animal species and will also depend upon the agonist or antagonist used, the rate of administration and the route of administration.

The adequacy of the vaccination parameters chosen, e.g. dose, schedule, adjuvant choice and the like, can be determined by taking aliquots of serum from the subject animal and assaying antibody titers during the course of the immunization program. For cell-mediated immunity, cytotoxic cell activity can be monitored using methods available in the art. The presence of immune T cells and their in vitro response to antigen can also be monitored. In addition, the clinical condition of the animal will be monitored for the desired effect, e.g. resistance to infection, anti-tumor activity or other effect. If inadequate vaccination is achieved then the animal can be boosted with further antigen vaccinations and the vaccination parameters can be modified in a fashion expected to potentiate the immune response, e.g. increase the amount of antigen and/or adjuvant, complex the antigen with a carrier or conjugate it to an immunogenic protein, or administer the antigen intraperitoneally.

In another embodiment, the invention provides purine receptor antagonists that can be used to decrease or diminish an undesirable immune response. For example, compositions and methods of the invention that include purine receptor antagonists can be used to diminish undesirable immune response against therapeutic agents and such antagonists can be used in the treatment of autoimmune diseases. Many autoimmune disorders are the result of inappropriate activation of T cells that are reactive against “self tissues” and that promote the production of cytokines and auto-antibodies involved in the pathology of the diseases. Modulation of immune cell activity by administration of the purine receptor antagonists of the invention can modulate or inhibit the course of the autoimmune disease.

Non-limiting examples of autoimmune diseases and disorders having an autoimmune component that may be treated according to the invention include diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

An effective amount of an antagonist of the invention for modulating (e.g. inhibiting) the immune system can be determined by the clinician taking into account the method and site of delivery of the antagonists of the invention, the desired form of the response (decreased antibody titer, decreased inflammation, or both), the presence of carriers and other considerations that will be apparent to those skilled in the art.

Further information on formulations and routes of administration contemplated by the invention is provided below.

Formulations and Administration

The compounds of the invention, including their salts, are administered so as to modulate the immune response. To achieve the desired effect(s), the compound, an analog thereof or a combination thereof, may be administered as single or divided dosages, for example, of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the compound chosen, the disease, the weight, the physical condition, the health, the age of the mammal, whether induction or diminution of an immune response is to be achieved, and if the compound is chemically modified. Such factors can be readily determined by the clinician employing animal models or other test systems that are available in the art.

Administration of the therapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the compounds of the invention may be essentially continuous over a preselected period of time or may be in a series of spaced doses.

To prepare the composition, compounds are synthesized or otherwise obtained, and purified as necessary or desired. In some embodiments, the compounds of the invention can be administered as prodrugs that are substantially inactive in prodrug form but can metabolized into active purine receptor agonists and antagonists. The compounds can also be lyophilized and/or stabilized. The compound can then be adjusted to the appropriate concentration, and optionally combined with other agents. The absolute weight of a given compound included in a unit dose can vary widely. For example, about 0.01 to about 2 g, or about 0.1 to about 500 mg, of at least one compound of the invention, or a plurality of compounds specific for a particular cell type can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the compounds of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

Thus, one or more suitable unit dosage forms comprising the therapeutic compounds of the invention can be administered by a variety of routes including topical, oral, parenteral (including subcutaneous, intravenous, intramuscular and intraperitoneal), rectal, dermal, transdermal, intrathoracic, intrapulmonary and intranasal (respiratory) routes. In general, topical, intradermal or subcutaneous administration is preferred.

The formulations may, where appropriate, be conveniently presented in discrete unit dosage forms and may be prepared by any of the methods well known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system. By “pharmaceutically acceptable” it is meant a carrier, diluent, excipient, and/or salt that is compatible with the other ingredients of the formulation, and not deleterious or unsuitably harmful to the recipient thereof. The therapeutic compounds may also be formulated for sustained release (for example, using microencapsulation, see WO 94/ 07529, and U.S. Pat. No.4,962,091).

Pharmaceutical formulations containing the therapeutic compounds of the invention can be prepared by procedures known in the art using well-known and readily available ingredients. For example, the compound can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatin, and polyvinyl-pyrrolidone. Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For topical administration, the therapeutic agents may be formulated as is known in the art for direct application to a target area. Forms chiefly conditioned for topical application take the form, for example, of creams, milks, gels, dispersion or microemulsions, lotions thickened to a greater or lesser extent, impregnated pads, ointments or sticks, aerosol formulations (e.g., sprays or foams), soaps, detergents, lotions or cakes of soap. Other conventional forms for this purpose include transdermal patches, wound dressings, coated bandages or other polymer coverings, ointments, creams, lotions, pastes, jellies, sprays, and aerosols. Thus, the therapeutic compounds of the invention can be delivered via transdermal patches or bandages for dermal administration. Alternatively, the compound can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 to about 200 microns.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active compounds can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-85% by weight.

Drops, such as eye drops or nose drops, may be formulated with one or more of the therapeutic compounds in an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic compound may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present invention in a suitable liquid carrier.

The therapeutic compounds of the invention can also be formulated as elixirs or solutions convenient for parenteral administration, for instance by intramuscular, subcutaneous, intraperitoneal or intravenous routes. The pharmaceutical formulations of the therapeutic compounds of the invention can also take the form of an aqueous or anhydrous solution or dispersion, or alternatively the form of an emulsion or suspension or salve.

Thus, the therapeutic compounds may be formulated for parenteral administration (e.g., by injection, for example, bolus injection or continuous infusion) and may be presented in unit dose form in ampoules, pre-filled syringes, small volume infusion containers or in multi-dose containers. As noted above, preservatives can be added to help maintain the shelve life of the dosage form. The active compounds and other ingredients may form suspensions, solutions, or emulsions in oily or aqueous vehicles, and may contain formulatory agents such as suspending, stabilizing and/or dispersing agents. Alternatively, the active compounds and other ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable carriers and vehicles that are available in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils and polysiloxanes.

It is possible to add other ingredients such as antioxidants, surfactants, preservatives, film-forming, keratolytic or comedolytic agents, perfumes, flavorings and colorings. Antioxidants such as t-butylhydroquinone, butylated hydroxyanisole, butylated hydroxytoluene and α-tocopherol and its derivatives can be added.

Additionally, the compounds are well suited to formulation as sustained release dosage forms and the like. The formulations can be so constituted that they release the active compound, for example, on the skin or in a particular part of the intestinal or respiratory tract, over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., prosthetic devices, pins, catheters, peritoneal dialysis tubing, draining devices and the like.

The pharmaceutical formulations of the present invention may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions pH 7.0-8.0.

The compounds of the invention can also be administered to the respiratory tract. Thus, the present invention also provides aerosol pharmaceutical formulations and dosage forms for use in the methods of the invention. In general, such dosage forms comprise an amount of at least one of the agents of the invention effective to treat or prevent the clinical symptoms of a specific infection, indication or disease. Any statistically significant attenuation of one or more symptoms of an infection, indication or disease that has been treated pursuant to the method of the present invention is considered to be a treatment of such infection, indication or disease within the scope of the invention.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatin or blister packs from which the powder may be administered with the aid of an inhalator, insufflator, or a metered-dose inhaler (see, for example, the pressurized metered dose inhaler (MDI) and the dry powder inhaler disclosed in Newman, S. P. in Aerosols and the Lung, Clarke, S. W. and Davia, D. eds., pp. 197-224, Butterworths, London, England, 1984).

Therapeutic compounds of the present invention can also be administered in an aqueous solution when administered in an aerosol or inhaled form. Thus, other aerosol pharmaceutical formulations may comprise, for example, a physiologically acceptable buffered saline solution containing between about 0.1 mg/ml and about 100 mg/ml of one or more of the compounds of the present invention specific for the indication or disease to be treated. Dry aerosol in the form of finely divided solid compound or nucleic acid particles that are not dissolved or suspended in a liquid are also useful in the practice of the present invention.

Compounds of the present invention may be formulated as dusting powders and comprise finely divided particles having an average particle size of between about 1 and 5 μm, alternatively between 2 and 3 μm. Finely divided particles may be prepared by pulverization and screen filtration using techniques well known in the art. The particles may be administered topically or by inhaling a predetermined quantity of the finely divided material, which can be in the form of a powder. It will be appreciated that the unit content of active ingredient or ingredients contained in an individual aerosol dose of each dosage form need not in itself constitute an effective amount for treating the particular infection, indication or disease since the necessary effective amount can be reached by administration of a plurality of dosage units. Moreover, the effective amount may be achieved using less than the dose in the dosage form, either individually, or in a series of administrations.

For administration to the upper (nasal) or lower respiratory tract by inhalation, the therapeutic compounds of the invention are conveniently delivered from a nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Nebulizers include, but are not limited to, those described in U.S. Pat. Nos. 4,624,251; 3,703,173; 3,561,444; and 4,635,627. Aerosol delivery systems of the type disclosed herein are available from numerous commercial sources including Fisons Corporation (Bedford, Mass.), Schering Corp. (Kenilworth, N.J.) and American Pharmoseal Co., (Valencia, Calif.). For intra-nasal administration, the therapeutic agent may also be administered via nose drops, a liquid spray, such as via a plastic bottle atomizer or metered-dose inhaler. Typical of atomizers are the Mistometer (Wintrop) and the Medihaler (Riker).

Furthermore, the active ingredients may also be used in combination with other therapeutic agents, for example, pain relievers, anti-inflammatory agents, antihistamines, and the like, whether for the conditions described or some other condition.

The present invention further pertains to a packaged pharmaceutical composition for modulating the immune system such as a kit or other container. The kit or container holds a therapeutically effective amount of a pharmaceutical composition for modulating the immune system and instructions for using the pharmaceutical composition for modulating the immune system. The pharmaceutical composition includes at least one compound of the present invention, in a therapeutically effective amount such that the immune system is modulated.

In another embodiment, the kit can include an antigen and a therapeutically effective amount of an adjuvant composition of the invention. The antigen and the adjuvant composition can be combined into one composition for administration together, or the antigen and adjuvant compositions can be packaged separately to permit separate administration. Instructions for administering the antigen and the adjuvant composition can also be included in the kit.

The invention is further illustrated by the following non-limiting Examples. It is known that extracellular nucleotides activate ligand-gated P2X receptor ion channels and G protein-coupled P2Y receptors. The Examples provided illustrate that intradermal administration of ATPγS, a hydrolysis-resistant P2 agonist, results in an enhanced contact hypersensitivity response. Furthermore, the Examples show that ATPγS enhanced the induction of immunity to a model tumor vaccine in mice and enhanced the antigen presenting function of Langerhans cells (LC) in vitro. Exposure of a LC-like cell line to ATPγS augmented the induction of I-A, CD80, CD86, IL-1β and IL-12 while inhibiting the expression of IL-10, suggesting that the immunostimulatory activities of purinergic agonists in the skin are mediated, at least in part, by P2 receptors on antigen presenting cells. These data indicate that ATP, when released after trauma or infection, may act as an endogenous adjuvant to enhance the immune response and P2 agonists may augment the immune response to vaccines.

### EXAMPLE 1

### Intradermal Administration of ATPγS Leads to an Exaggerated Contact Hypersensitivity Response

This Example shows that mice immunized with an antigen (dinitro-fluorobezene) and ATPγS exhibit heightened contact hypersensitivity (CHS) responses.

Mice and Cells

BALB/c (H-2^{d}) and CAF₁ (H-2^{a/d}) mice were obtained from the Jackson Laboratory. All experiments were performed in compliance with relevant laws and institutional guidelines and were approved by the Weill Medical College Institutional Animal care and Use Committee. XS106 cells were derived from neonatal A/J epidermis as described in Matsue, H. et al. Induction of antigen-specific immunosuppression by CD95L cDNA-transfected “killer” dendritic cells. Nat. Med. 5, 930-937 (1999).

Reagents

ATP, ATPγS and KLH were obtained from the Sigma Chemical Company (St. Louis, Mo.).

Contact Hypersensitivity (CHS) Assay

Groups of 5 BALB/c mice were injected intradermally on the back with 0.05 ml of PBS or PBS containing 30 nanomoles of ATPγS (a stable analog of ATP). Fifteen minutes later these mice were immunized by application of 5 μl 1% dinotrofluorobenzene (DNFB) in acetone:olive oil (4:1) at the injected site. Control mice were injected with medium or ATPγS but were not immunized with DNFB. Seven days later all mice were challenged on the ears by application of 5 μl of 0.2% DNFB in acetone to each side of each ear. Ear swelling was assessed with an engineer's spring-loaded micrometer before challenge and 24 or 48 hours after challenge as described in Ding, W. et al. Altered cutaneous immune parameters in transgenic mice overexpressing viral IL-10 in the epidermis. J. Clin. Invest. 111, 1923-1931 (2003). The pre-challenge ear thicknesses were subtracted from the value observed 24 or 48 hours after challenge and ear swelling used as a measure of CHS response.

Statistical Analysis

The significance of differences amongst groups was measured by Students' two-tailed t-test for unpaired samples (Excel software, Microsoft, Redmond Wash.).

Results: ATPγS Enhances the Induction of Contact Hypersensitivity

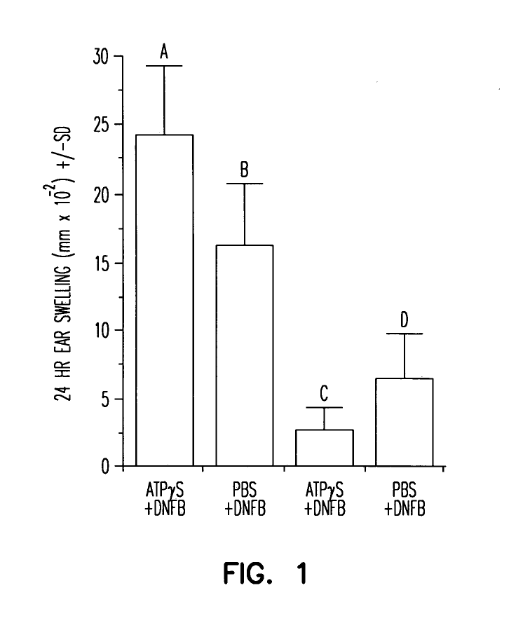

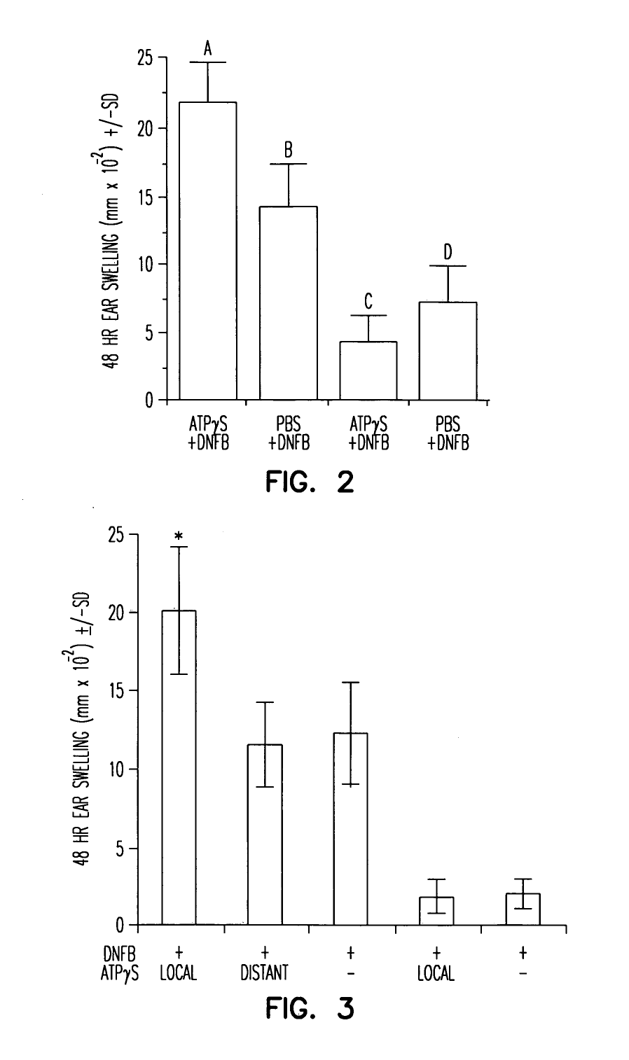

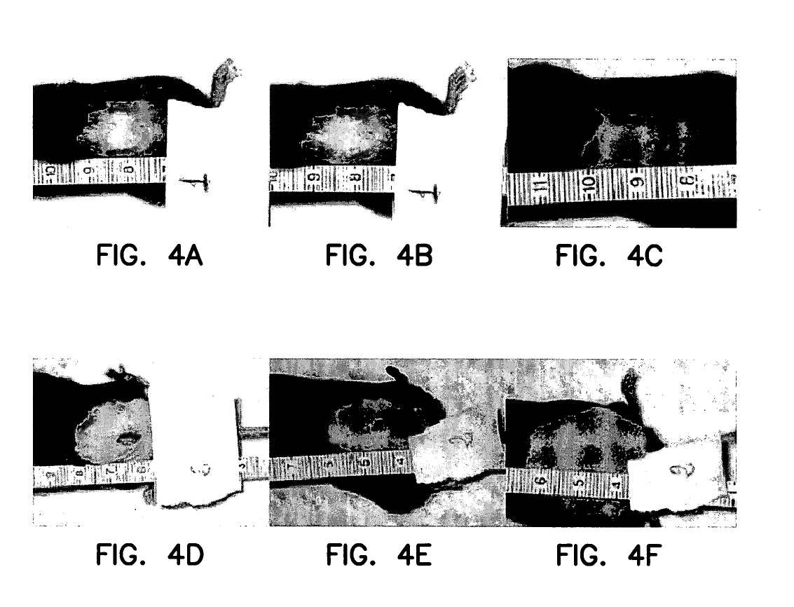

As shown by the data in FIGS. 1 and 2, intradermal administration of ATPγS significantly augmented the contact hypersensitivity response at 24 and 48 hours after administration, respectively, indicating a striking regulation of cutaneous immunity by ATP. Moreover, as shown in FIG. 3, intradermal administration of ATPγS at the site of immunization (but not at a distant site) significantly increased the contact hypersensitivity response, supporting the hypothesis that P2 agonists enhance immune responsiveness.

### EXAMPLE 2

### Purine Receptor Agonists Enhance Immunization Against Group A Streptococcus (Streptococcus pyogenes) Infection

To determine whether ATP augments the efficacy of a vaccine, a well-established model of murine infection with group A streptococcus (GAS) was employed. Nizet et al., Nature 414:454-57 (2001). Groups of three C57BL/6 mice were injected intradermally with 10⁸ killed (freeze-thawed×4) GAS in 100 μl of medium containing 700 nanomoles of ATPγS or GAS in medium alone (without ATPγS). Immunizations were repeated twice at 7 day intervals. One week after the last immunization all mice were shaved, hair removed by chemical depilation with topical sodium thioglycollate and then injected subcutaneously with 50 μl of a mid-logarithmic growth phase (A₆₀₀=0.6, about 5×10⁷ c.f.u.) of live GAS complexed to Cytodex beads as a carrier. Lesion sizes were measured daily. At day 5, mice immunized without ATPγS had a mean lesion size of 25.01±10.35 compared to 2.80±1.11 mm² for mice immunized with ATPγS (p=0.021, student's two-tailed t-test for unpaired values).

The significance of differences amongst groups was measured by Students' two-tailed t-test for unpaired samples (Excel software, Microsoft, Redmond Wash.).

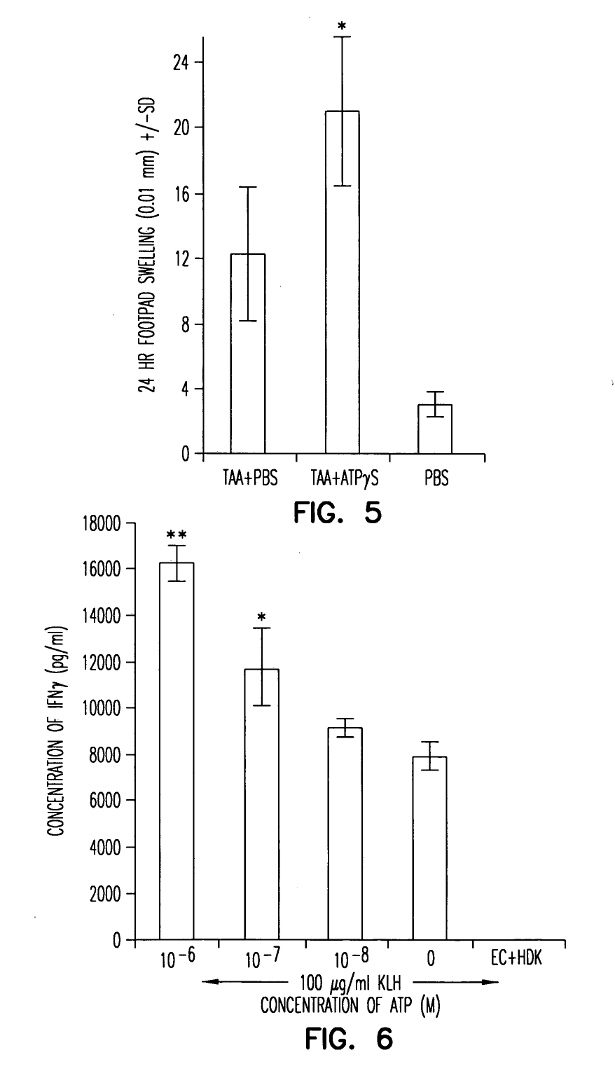

As shown in FIG. 4, mice immunized with killed GAS in the presence of ATPγS (top three panels) developed much smaller lesions than mice immunized with killed GAS alone (bottom three panels). These data indicate that purinergic agonists are useful immunologic adjuvants.

### EXAMPLE 3

### Purine Receptor Agonists Enhance Immunization Against Tumor-Associated Antigens

This Example shows that addition of ATPγS to a model anti-tumor vaccine enhances the immune response of mice against tumor antigens.

Tumor Vaccine

Groups of 5 CAF₁ mice were injected intradermally with 0.1 ml of a soluble extract of tumor-associated antigens (TAA) from the S1509a spindle cell tumor (derived from A/J, H-2^{a}) as described in Grabbe, S. et al. Tumor antigen presentation by murine epidermal cells. J. Immunol. 146, 3656-3661 (1991). Test groups of mice injected with tumor-associated antigens also received 30 nanomoles of ATPγS, while negative control groups of mice received PBS and no ATPγS. TAA were prepared by suspending S1509a cells in PBS at a concentration of 10⁷/ml, subjecting the suspension to 4 cycles of freeze-thawing and harvesting the supernatant after centrifugation at 600×g. One week after immunization, each mouse was challenged in a hind footpad by injection with 50 μl of TAA and 24 hour swelling assessed by measuring the hind footpads with a spring-loaded micrometer and subtracting the thickness of the uninjected footpad from that of the injected footpad.

Statistical Analysis

The significance of differences amongst groups was measured by Students' two-tailed t-test for unpaired samples (Excel software, Microsoft, Redmond Wash.).

Results: ATPγS Augments Immunity to a Model Tumor Vaccine

To examine whether ATPγS enhances immunity induced by a vaccine, CAF₁ mice were immunized by intradermal injection of tumor-associated antigens (TAA) or TAA containing 30 nanomoles of ATPγS. Negative control mice were not immunized.

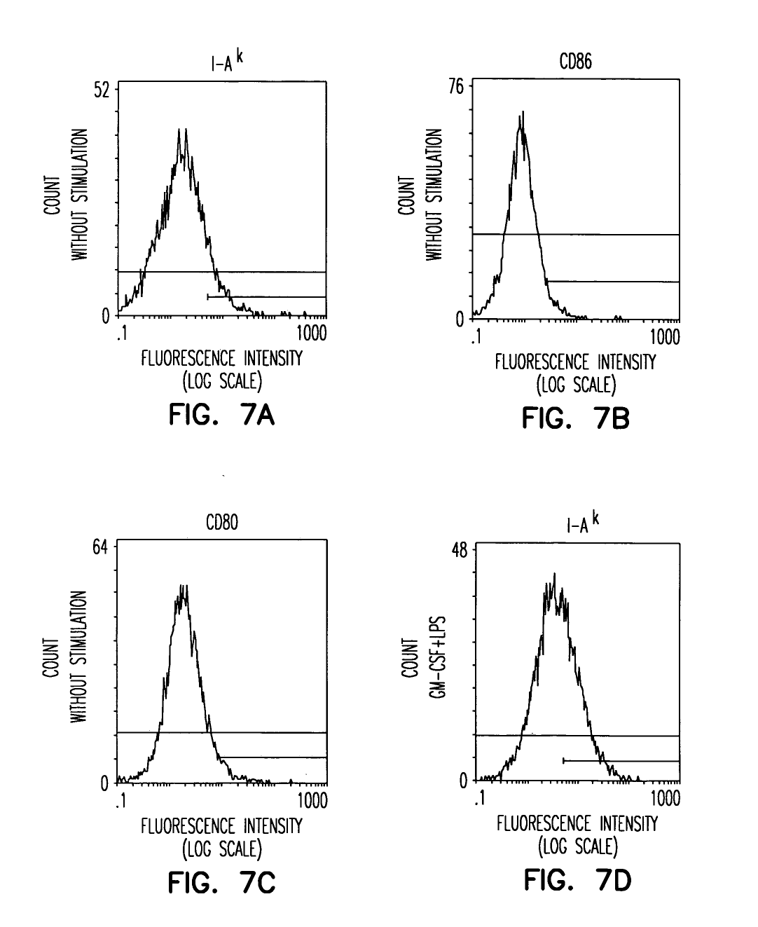

One week after the last immunization, each mouse was challenged in a hind footpad with TAA. As shown by the data in FIG. 5, ATPγS significantly enhanced the delayed-type hypersensitivity response at 24 hours.

### EXAMPLE 4

### ATPγS Augments Antigen Presentation to T cells by Langerhans Cells

This Example illustrates that ATPγS increases antigen presentation by Langerhans cells.

HDK-1 Assay

BALB/c epidermal cells were prepared using a modification of the protocol described in Grabbe, S. et al. Tumor antigen presentation by murine epidermal cells. J. Immunol. 146, 3656-3661 (1991). Briefly, mice were shaved with an electric clipper and were depilated with topical sodium thioglycolate. The mice were then euthanized and truncal skin excised. Subcutaneous fat and paniculus carnosus were mechanically removed. The epidermis was separated from the dermis by floating skins on 0.5 units/ml dispase and 0.38% trypsin in PBS for 45 minutes. Epidermal sheets were then washed and a single cell suspension was prepared by mild agitation. Langerhans cell (LC) content was enriched by treatment of epidermal cells with monoclonal anti-Thy 1.2 antibodies followed by low toxicity rabbit complement (Cedarlane, Homby, Ontario). The cells were then incubated with anti-I-A^{d} antibody (BD Pharmingen, San Diego, Calif.) followed by incubation with goat anti-mouse IgG conjugated to magnetic microspheres (DynaBeads -450; Dynal Biotech, Lake Success, N.Y.). Cells bound to antibody were separated using the magnetic field. By FACS analysis these cells were more than 95% LCs.

Langerhans cells were plated in 96 well plates at 1×10⁴ cells per well and incubated with KLH (50 μg/ml) for 2 hours. Then ATPγS was added and cells cultured for an additional 3 hours. The cells were then washed three times and co-cultured with 5×10⁴ HDK-1 cells. Supernatants were collected after 72 hours for measurement of IFNγ by ELISA.

Statistical Analysis

The significance of differences amongst groups was measured by Students' two-tailed t-test for unpaired samples (Excel software, Microsoft, Redmond Wash.).

Results: ATPγS Augments Antigen Presentation by Epidermal Langerhans Cells

To determine whether ATPγS increases the antigen presenting capability of Langerhans cells (LCs), antigen presentation to the T cell clone HDK-1 was examined in the presence of ATPγS. This cloned T cell line responds to presentation of keyhole limpet hemocyanin (KLH) by I-A^{d} with production of gamma interferon (IFNγ). A population of enriched LCs was cultured in medium alone or medium containing graded concentrations of ATPγS. After 3 hours, KLH was added and 2 hours later cells were gamma irradiated and washed 3 times to remove any residual ATPγS. LCs were then co-cultured with HDK-1 cells. After 72 hours, supernatants were harvested and IFNγ content was measured by ELISA.

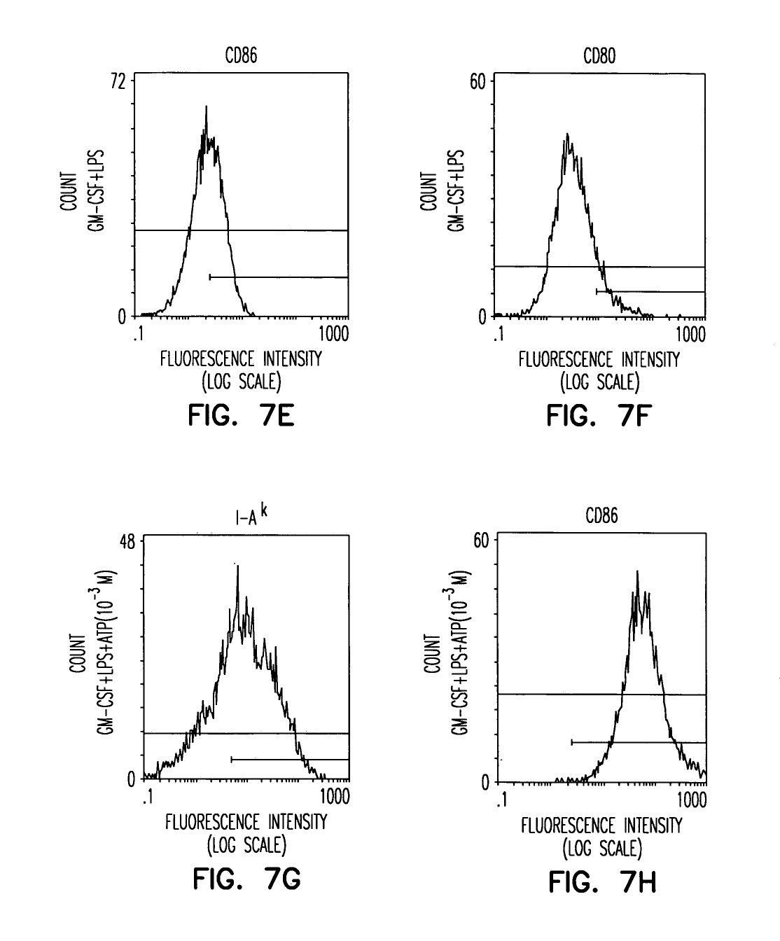

Exposure to ATPγS resulted in a dose-dependent increase in IFNγ release (FIG. 6). When LCs were incubated with ATPγS in the absence of KLH, only background levels of IFNγ were produced. Thus, ATPγS enhanced antigen presentation. At concentrations greater than10⁻⁶ molar the response begins to decrease, possibly due to induction of apoptosis as concentrations higher than 10⁻⁶ molar induce apoptosis in the LC-like cell line XS106 (data not shown). These data demonstrate that ATPγS increases the ability of Langerhans cells to present antigen to T cells in vitro.

### EXAMPLE 5

### ATPγS Augments Expression of I-A, CD80 and CD86 in XS106A Cells

This Example illustrates that purinergic agonists enhance the expression of I-A^{k}, CD80, and CD86.

Cytokine and FACS Analysis

XS106 cells were plated in 12 well plates at a concentration of 1×10⁶ cells/ml in RPMI medium containing 10% fetal calf serum, 100 units/ml penicillin, 100 mg/ml streptomycin 0.1 mm non-essential amino acids, 0.1 mm essential amino acids, 2 mm L-glutamine, 1 mm sodium pyruvate, and 10 mm Hepes buffer (all from Cellgro, Herndon, Va.). Cells were stimulated by lipopolysaccharide 1 μg/ml and 50 units/ml of GM-CSF (or GM-CSF alone) in the presence or absence of graded concentrations of ATPγS. After 48 hours cells were harvested for FACS analysis and supernatants for analysis of cytokine content by ELISA. For FACS analysis, cells were stained with FITC-conjugated anti-mouse I-A^{d} (BD Pharmingen, San Diego, Calif.), PE-conjugated anti-mouse CD86 (BD Pharmingen, San Diego, Calif.) or PE-conjugated anti-mouse CD80 (BD Pharmingen, San Diego, Calif.).

As an additional approach to test the importance of purine receptors, the ability of oxidized ATP (oATP) to inhibit the effect of ATPγS on up-regulation of CD80 by ATPγS was tested. oATP is an irreversible inhibitor of P2X₇. It is currently unclear whether it has activity at other P2X receptors but it does not appear to block P2Y receptors. Di Virgilio et al., Blood 97:587-600, 2001. The structure of oxidized ATP is shown below. 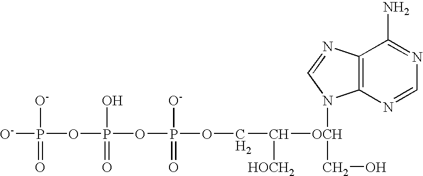
Cells were pre-incubated with 300 μM oATP for 2 hours prior to extensive washing and stimulation with GM-CSF/LPS and ATPγS as described above. ps Statistical Analysis

The significance of differences amongst groups was measured by Students' two-tailed t-test for unpaired samples (Excel software, Microsoft, Redmond Wash.).

Results: ATP Agonists Augment Expression of I-A, CD80 and CD86

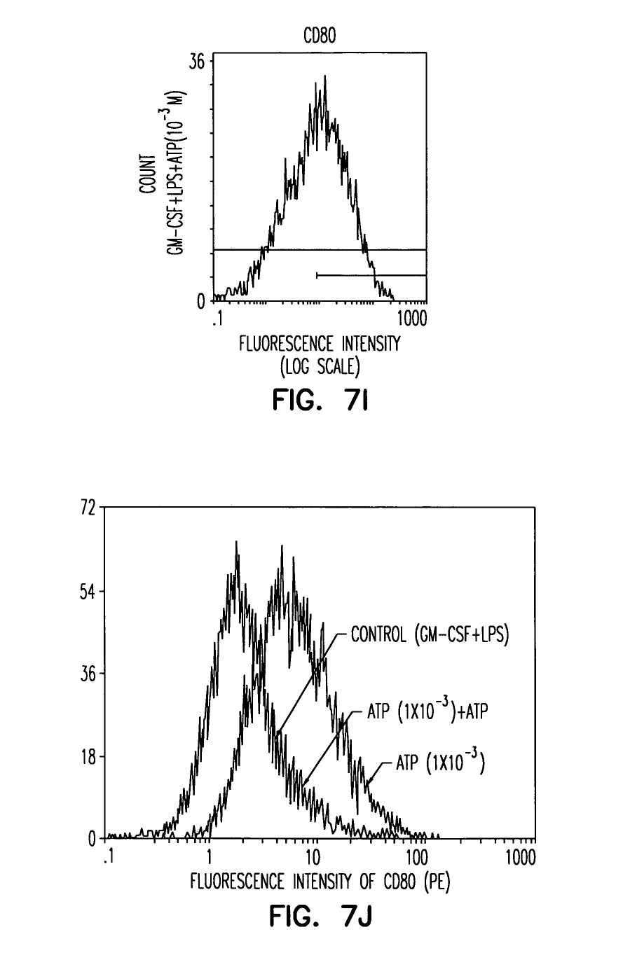

To explore mechanisms by which ATP agonists produce the above effects, studies were conducted on the effect of ATPγS on XS 106, a cloned cell line that was derived from neonatal A/J epidermis and has many of the functional and phenotypic characteristics of LCs. See Matsue, H. et al. Induction of antigen-specific immunosuppression by CD95L cDNA-transfected “killer” dendritic cells. Nat. Med. 5, 930-937 (1999). To stimulate expression of I-A, CD80 and CD86, XS106 cells were cultured in medium containing GM-CSF and LPS in the presence of graded concentrations of ATPγS. As indicated by FACS analysis, expression of three surface molecules that are important for antigen presentation (I-A, CD80 and CD86) were enhanced by exposure to ATP (FIG. 7A-I). Because this is a cell line, any effects observed were presumably due to direct effects on the cells. These data indicate that ATPγS can directly modulate LC maturation.

The enhanced expression of CD80 by ATPγS was inhibited by oATP (FIG. 7J). Thus, ATP promotes the maturation of cell lines of Langerhans-like cells and increases the expression of cell surface molecules that are involved in antigen presentation.

### EXAMPLE 6

### Purine Receptor Agonists Stimulate IL-1β Expression and Reduce IL-10 Expression

Another way that ATP or ATPγS might contribute to antigen presentation is by altering the expression of cytokines. This Example illustrates that exposure to ATP or ATPγS stimulates expression of cytokines IL-1β and IL-12 (cytokines that promotes contact hypersensitivity) but decreases expression of IL-10 (a cytokine that dampens cell-mediated immune responses).

Methods

XS 106A cells were cultured in medium containing 10 U/ml of GM-CSF and 0.1 μg/ml of LPS in the presence or absence of increasing concentrations of ATP or ATPγS. After 24 hours, supernatants were harvested and cytokine content was measured by ELISA.

OPtEIA™ sets of capture antibody and biotinylated detection monoclonal antibody for murine were purchased from BD Pharmingen (San Diego, Calif.). One hundred μl of capture antibody (4 μg/ml. in coating buffer) was added to each well in 96-well flat-bottom plates and incubated overnight at 4° C. Each well was then aspirated and washed 3 times with PBS-Tween. Wells were then blocked with 200 μl of 10% fetal bovine serum in PBS followed by aspirating and washing each well 3 times with PBS-Tween. One hundred μl of each standard and sample was then added to wells. After incubation at room temperature for 2 hrs, each well was aspirated and washed 5 times with PBS -Tween. One hundred microliters of detection antibody (1 μg/ml) was added to each well followed by incubation at room temperature for 1 hour. Wells were aspirated and washed 5 times with PBS-Tween. One hundred microliters of Avidin-HRP (1:2000) was added to each well followed by incubation for 30 min at room temperature. After aspiration and washing of each well 7 times with PBS-Tween, 100 μl of substrate solution (tetramethylbenzidine and hydrogen peroxide) was added to each well at room temperature in the dark. Fifty microliters of stop solution (1M H₃PO₄) was added to each well and wells were read at 450 nm.

Results: ATP Agonists Enhance IL-1β and IL-12 Secretion and Reduce IL-10 Release

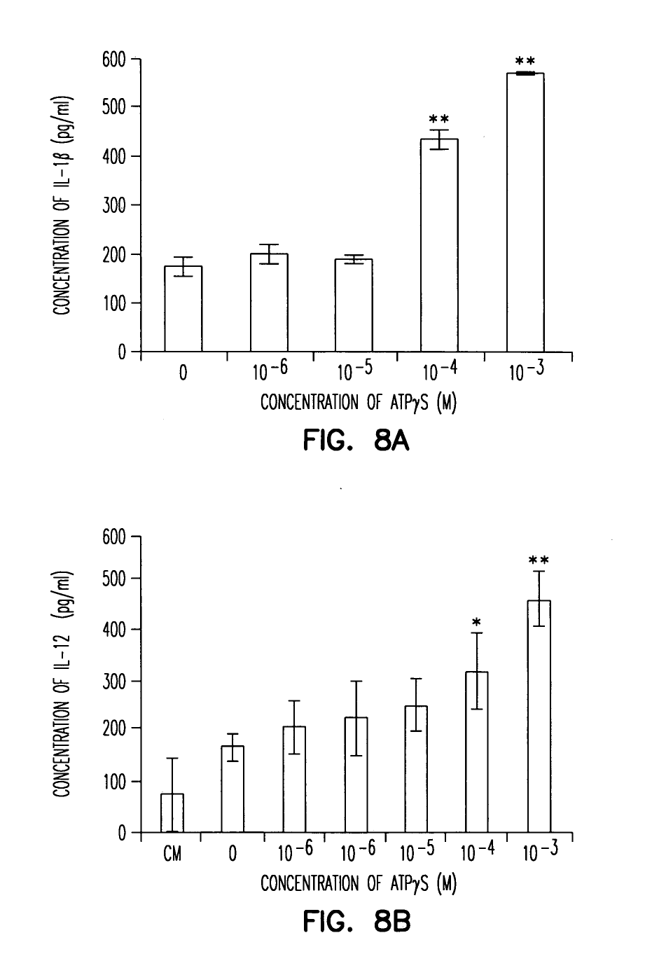

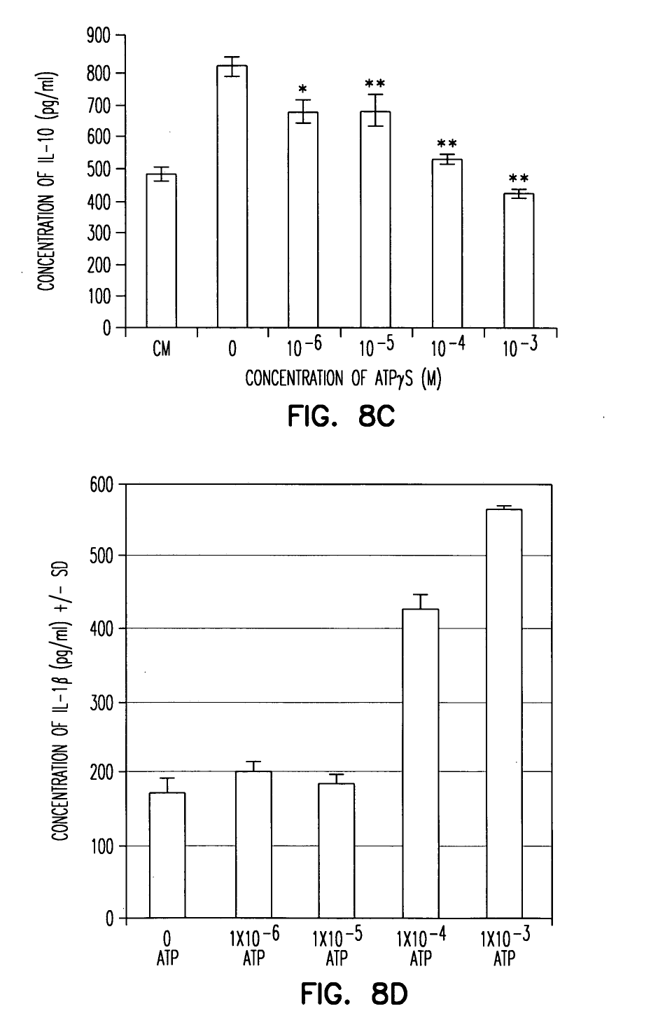

ATPγS significantly increased release of IL-1β and IL-12 while decreasing release of IL-10 from GM-CSF/LPS-stimulated XS 106 cells (FIG. 8A-C). ATP also greatly augmented secretion of IL-1β (FIG. 8D) at a variety of concentrations. Oxidized ATP (oATP), a purinergic receptor-antagonist, significantly inhibited the effect of ATPγS on IL-1β secretion (data not shown). Thus, the effects of ATP agonists on immune responsiveness may be regulated by cytokine expression by antigen presenting cells in the skin.

### EXAMPLE 7

### Nanomolar ATP Induces Little Langerhans Cell Apoptosis

ATP can induce apoptosis of some cell types. It is possible that a decrease in cytokine production (IL-10) could be caused by cell death. This is unlikely because ATP causes an increase in IL-1β concentration, so cell death is probably not a significant effect. Furthermore, for other cell types, induction of apoptosis by ATP occurs at higher concentrations than those used here, usually in the micromolar range.

To address this question more directly, XS 106A cell viability was examined of by Trypan blue exclusion using concentrations of ATP up to one micromolar. No effect on viability was seen up to 48 hours of culture (data not shown). As an additional test to determine whether increased cytokine release could explain the increase in cytokines, 1×10⁵ XS106A cells were cultured in 200 μl of complete medium without stimulation. The cells were disrupted by freeze-thaw lysis and the supernatant was assayed for IL-1β and IL-10 content. The concentration of these cytokines was below the limits of detection for the ELISA assays employed in our laboratory.

Thus, changes in cytokine content of supernatants from XS106A cells treated with ATP do not appear to relate to induction of cell death.

### EXAMPLE 8

### Langerhans-like Cells Express Purine Receptor mRNA

By analogy with monocyte-derived dendritic cells, Langerhans cells should express purine receptors. As illustrated herein, this expectation is supported by the responsiveness of these cells to ATP and ATPγS (see above). As an additional test, RT-PCR was used to determine weather XS 106A cells express mRNA for selected purine receptors. ps RT-PCR Assay

XS106A cells were used as a surrogate for Langerhans cells in these experiments. XS106A is a dendritic cell line derived from neonatal A/J (H-2^{a}) epidermis that has many phenotypic characteristics of Langerhans cells.

The following P2Y₁, P2Y₂, P2Y₄, P2Y₅, P2Y₆, P²Y₁₀ and P2Y₁₁ primers were designed from mouse sequences in Genbank:

| | | | |
|---|---|---|---|
| P2X1, | 5′-CATTGTGCAGAGAACCCAGAA-3′ | (SEQ ID NO:1) | forward |
| and | | | |
| | 5′-ATGTCCTCCGCATACTTGAAC-3′ | (SEQ ID NO:2) | reverse primer; |
| P2X7, | 5′ GGCAGTTCAGGGAGGAATCATGG-3′ | (SEQ ID NO:3) | forward, |
| and | | | |
| | 5′-AAAGCGCCAGGTGGCATAGCTC | (SEQ ID NO:4) | reverse primer; |
| P2Y1, | 5′-AGCAGTTTCCTCTTCATTCC-3′ | (SEQ ID NO:5) | forward, |
| and | | | |
| | 5′-CCGAGTCCCAGTGCCAGAGT-3′ | (SEQ ID NO:6) | reverse primer; |
| P2Y2, | 5′-CGTGGGGGAGAGTAGTGTAG-3′ | (SEQ ID NO:7) | forward, |
| and | | | |
| | 5′-GGGAGGGGAGATAGAGGAGA-3′ | (SEQ ID NO:8) | reverse primer; |
| P2Y4, | 5′-AGCCCAAGTTCTGGAGATGGTG-3′ | (SEQ ID NO:9) | forward, |
| and | | | |
| | 5′-GGTGGTTCCATTGGCATTGG-3′ | (SEQ ID NO:10) | reverse primer; |
| P2Y9, | 5′-ACCATCAGGCCAGGAGGAA-3′ | (SEQ ID NO:11) | forward, |
| and | | | |
| | 5′-ACAAAGCATACCACAAACAC-3′ | (SEQ ID NO:12) | reverse primer; |
| P2Y11, | 5′-TGTATTCTTTATTTGCTTTG-3′ | (SEQ ID NO:13) | forward, |
| and | | | |
| | 5′-CGTCCCTTTGGTTTTGTTGT-3′ | (SEQ ID NO:14) | reverse primer. |

Briefly, 0.1 μg of total RNA from XS106A cells was incubated in a reaction mixture containing 5 mM MgCl₂, 50 mM KCl, 10 mM Tris-HCL, 1 mM dNTPs, 2.5 μM random hexamers, 1 U RNAse inhibitor/μl and 2.5 U M-MLV reverse-transcriptase/μl in 20 ul for 10 minutes at room temperature for the extension of the hexameric primers, for 15 minutes at 42° C. for annealing and then for 5 minutes at 99° C. to denature the enzyme. Twelve μl of the reverse-transcribed reaction was transferred to a PCR reaction mixture for the receptors and 6 μl of the reverse-transcribed reaction was transferred from the same tube to a PCR reaction mixture for GAPDH as an internal control. The reaction mixture consisted of 50 mM KCL, 10 mM Tris-HCL, 2 mM MgCl₂, 0.5 μM primers and 1 U AmpliTaq DNA polymerase in a total volume of 40 μl. After denaturation for five minutes at 95° C., 25-40 cycles of denaturation for 30 seconds at 95° C., annealing for 30 seconds at 55° C. and extension for 30 seconds at 72° C. was repeated with a thermal cycler followed by completion for 50 minutes at 72° C. The PCR products were electrophoresed in 1% agarose gel, stained with ethidium bromide and visualized with ultraviolet radiation.

Results: XS106 Cells Express mRNA for P2 Receptors

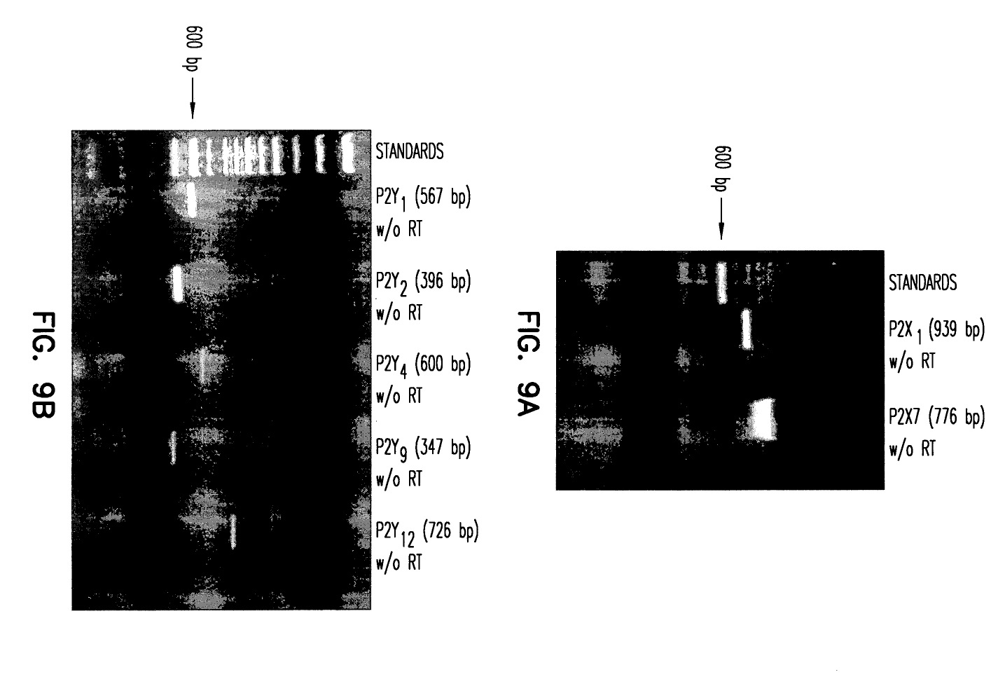

All of the experiments described above are consistent with the hypothesis that ATP acts as an adjuvant by binding to cell surface P2 receptors. As a direct test for the presence of these receptors, reverse transcription-polymerase chain reaction (RT-PCR) was used. XS106 cells were found to express mRNA for P2X1, P2X7, P2Y1, P2Y2, P2Y4, P2Y9 and P2Y11 receptors (FIG. 9A-B). In each case the PCR product was of the predicted length and was dependent on the presence of reverse transcriptase. Thus, LCs express several different P2 receptors.

### EXAMPLE 9

### Immunization Against Antigens

Experiments will be performed to determine if ATPγS enhances immunization against other antigens such as ricin (a category B toxin with potential as an agent of bioterrorism) and HIV. Further studies on the effects of ATPγS during immunization against group A streptococcus (GAS) and against tumor-associated antigens such as S1509a tumor cells will also be performed.

Vertebrate Animals

Animals that can be tested for immunization against a variety of antigens include CAF₁, BALB/c and C57BL/6 mice. Mice used in these studies will be about 8 to 12 weeks old. The use of such mice allows for ready interpretation of the results of immunologic manipulations.

Immunization Against Ricin

Ricin toxin (RT) is a 64 kDa protein found in castor beans. The toxin consists of two polypeptide chains (A and B) connected by a disulfide bond. The A chain (RTA) is a ribotoxin which inhibits mammalian cell protein synthesis. The B chain is a lectin capable of binding to galactose on cell surfaces. The ricin toxin A chain enzymatically inactivates the 60 S ribosome and a single molecule in the cytoplasm of a cell can completely inhibit protein synthesis. The estimated lethal dose of RT in humans is 1 to 10 μg/kg. Because of its great toxicity, RT represents a potential biological warfare agent.

Three RT preparations are available for vaccination. One is formalinized RT while the second is deglycosolated RTA. Although these preparations are effective, it is difficult to completely inactivate RT with formalin and deglycosolated RTA still has risk for systemic vascular leak syndrome. We will examine the ability of ATP and ATPγS to augment the antibody response to mutant A chain subunits of ricin (RTAms) which has been modified to remove toxic activity but retain antigenicity. Colleagues have generated two RTAms containing a mutation in the protein's enzymatic site, yielding proteins that lack toxicity as compared to the wild-type RTA. Smallshaw J E, Firan A, Fulmer J R, Ruback S L, Ghetie V, Vitetta E S. A novel recombinant vaccine which protects mice against ricin intoxication. Vaccine 20:3422-3427, 2002. This group has demonstrated that vaccination of BALB/c mice with these RTAms results in induction of a significant and measurable antibody response and protection against challenge with an intraperitoneal injection of a lethal dose of ricin toxin.

Groups of five BALB/c mice will be injected with 0.1, 0.25 or 0.5 μg/gm mouse of each RTAm or PBS alone weekly for each of four weeks. Groups of five experimental mice will be injected similarly RTAm (0.1, 0.25 or 0.5 μg/gm) except that the RTAm will be dissolved in medium containing 300 nanomoles of ATPγS. An additional set of mice will be immunized with RTAm dissolved in PBS containing 1000 nanomoles of ATP.

One week after the last injection, all mice will be challenged by an intraperitoneal injection of 100 ng/gm mouse of RT, a dose representing ten-fold the LD₅₀ for RT. The weights and deaths of animals will be recorded daily for 10 days, a period of time necessary for almost all vaccinated animals to regain their initial weight. Mice will also be bled retroorbitally before vaccination, before the administration of RT and one week after challenge with RT. Sera will be collected to determine the concentration of anti-RTA antibody as described below.

Ninety-six well plates will be coated at 4° C. with 100 μl wild-type RTA or recombinant RTAm at 20 μg/ml in PBS. After washing and blocking the plates with 5% FCS, 100 μl of mouse anti-RTA antibody at concentrations ranging from 1 to 1000 ng/ml (standard curve) or 100 μl of sera from vaccinated or control mice at various dilutions will be added in triplicate. After overnight incubations at 4° C., the plates will be washed and I-125-labeled rabbit anti-mouse Ig will be added. After two hours of incubation at room temperature, the plates will be washed and radioactivity in the wells counted in a gamma counter.

Ninety-six well plates will be coated at 4° C. with 100 μl wild-type RTA or recombinant RTAm at 20 μg/ml in PBS. After washing and blocking the plates with 5% FCS, 100 μl of mouse anti-RTA antibody at concentrations ranging from 1 to 1000 ng/ml (standard curve) or 100 μl of sera from vaccinated or control mice at various dilutions will be added in triplicate. After overnight incubations at 4° C., the plates will be washed and I-125-labeled rabbit anti-mouse Ig will be added. After two hours of incubation at room temperature, the plates will be washed and radioactivity in the wells counted in a gamma counter.

Immunization and Protection Against GAS (Streptococcus pyogenes)

This work is intended to be an extension of the studies already performed on mice using GAS and ATPγS (see Example 2 and FIG. 4). Groups of 5 C57BL/6 mice will be injected intradermally with 10⁸ killed (freeze-thaw×4) GAS in 100 μl containing 0, 50, 100 or 300 nanomoles of ATPγS; 0, 100, 300 or 1000 nanomoles of ATP or medium alone (without bacteria). Immunizations will be repeated twice at 7 day intervals. One week after the last immunization all mice will be shaved and hair removed by chemical depilation with topical sodium thioglycollate and then injected subcutaneously with 50 μl of a mid-logarithmic growth phase (A₆₀₀=0.6,˜5×10⁷ c.f.u.) of GAS complexed to Cytodex beads as a carrier. Lesion sizes will be measured daily. If ATP or ATPγS acts as an adjuvant, then groups vaccinated with these should have decreased lesion size.

Tumor Immunization

Groups of 5 näive CAF₁ mice will be immunized as follows: Groups will be injected intradermally with 30 μl of TAA mixed with 50, 100 or 300 nanomoles of ATPγS. Other groups of mice will be immunized with 30 μl of TAA plus 100, 300 or 1000 nanomoles of ATP. An additional group will be immunized by 30 μl of TAA alone; and control groups will be “immunized” with ATPγS, ATP and medium alone, without TAA. One week later all mice will be challenged by subcutaneous injection on the flank with 2×10⁶ live S1509a cells. Tumor growth will be scored every 48 hours by taking the product of three perpendicular tumor diameters. If either ATP or ATPγS is an effective adjuvant, then there will be a reduction of tumor growth in these groups.

Humoral Response to gp120

Immunization experiments will also be performed using the HIV envelope protein gp120 as antigen. Animals will be immunized and the humoral response will be determined. As a positive control the purified triterpene glycoside derivative of Quillaia saponins, QS21, will be utilized. QS21 has been shown to augment cellular responses and IgG subclass switching to a predominant IgG_{2A} subclass when employed as a vaccine adjuvant. Moore et al, Vaccine 17:2517-2527, 1999. It has been used with a range of viral antigens in both murine and non-human primate models without toxic side effects.

Groups of five BALB/c (H-2^{d} mice) will be immunized intradermally as followed. Groups of mice will receive 10 μg of recombinant gp120 mixed with 50, 100 or 300 nanomoles of ATPγS. A second set of groups will receive 10 μg of gp120 mixed with 100, 300 or 1000 nanomoles of ATP. A third group will receive 10 μg of gp120 mixed with 10 μg of QS21 (positive control). A fourth group will receive 10 μg of gp120 without adjuvant. Negative control groups will be immunized with each adjuvant dose or medium alone without gp120. This immunization schedule will be repeated at three weeks after initial immunization and again at nine weeks after initial immunization. Three weeks after the last immunization, mice will be euthanized, blood collected and serum prepared for evaluation of antibody responses as described below. Both the antibody titer and the antibody class will be determined.

Additionally, spleens will be harvested from all mice, a single cell suspension of nucleated spleen cells prepared, and cultures set up as below for determination of cytokine profile.

Serum samples will be tested for anti-gp120 IgM and IgG subclasses by ELISA. Serial two-fold dilutions of serum will be added to microtiter plates coated with recombinant gp120 (1.0 μg/ml) and bound antibodies detected using alkaline phosphatase-conjugated antibodies specific for whole IgM, whole IgG, or for IgG subclasses (PharMingen, San Diego, Calif. U.S.A.). The results will be expressed as endpoint titers calculated by regression of the straight part of a curve of OD versus serum dilution to a cutoff of two standard deviations above background control values.

To assess the cytokine profile elicited by gp120, spleen cells from each of the groups described above will be re-suspended at 2×10⁶ cells/ml in RPMI containing 10% fetal calf serum. Cells will be cultured in triplicate in 96 well round bottom microtiter plates in the presence of soluble HIV gp 120 protein (5 μg/ml), medium alone, or concanavalin A (2.0 μg/ml). A proliferative response will be assessed by pulsing each well with 1 μg of tritiated thymidine on the fourth day of culture and harvesting cells after 12 hours. Incorporation of radioactivity into acid-precipitable material will be assessed in each well by beta-scintillation counting. Supernatants from a duplicate culture plate will be removed 24 hours after setting up cultures to assess IL-2 production and after 72 hours to test for IL-4, IL-5, and IFNγ production. The concentrations of each of these cytokines will be determined by specific enzyme linked immunoabsorbent assays using commercially available antibodies (PharMingen). The mean cytokine concentrations in supernatants from triplicate cultures will assessed from a standard curve prepared with recombinant cytokines of known concentration.

Interpretation of Results

The significance of differences between groups in the GAS experiments will be assessed by multiple regression analysis. The significance of differences in antibody content amongst groups will be tested by Student's two-tailed t-test for unpaired values. With regard to gp120, gp120 alone will elicit a poor response, but that QS21 should increase antibody titer. The question asked is whether ATPγS acts as an adjuvant for a humoral response and if so, how it modifies the cytokine profile of stimulated cells. The significance of differences in tumor growth rates will be tested by examination of the means of the slope of tumor growth in each mouse (using a “best-fit” model that assumes linear growth rate) by Student's two-tailed t-test for unpaired values. The significance of differences in the protection against ricin intoxication experiments will be assessed by Kaplan-Meier statistics. All experiments will be repeated for verification of results. Thus, each of these experiments will test the effectiveness of purinergic receptors in promoting a biologically important response.

The experiments with ricin and streptococcus will test whether ATP increases the effectiveness of vaccination against these agents. Educated guesses will be made about the dose of adjuvant and vaccination schedule to be used in these experiments. If negative results are obtained, then additional experiments examining a broader range of ATP/ATPγS doses will be performed. Should tumor growth be totally or partially inhibited by immunization with TAA in the presence of ATP or ATPγS compared to mice immunized with TAA alone, it would be interpreted as showing an adjuvant effect of ATP or ATPγS. If one immunization is insufficient to observe the induction sufficient immunity to inhibit tumor growth, then two cycles of immunization can be tried in the same manner. As above, if no effect of ATP or ATPγS is observed, additional experiments examining a broader range of ATP/ATPγS doses will be performed.

References:

1. Beissert S, Granstein R D. UV-induced cutaneous photobiology. Crit Rev Biochem Mol Biol 1:381-404, 1996.
2. Grabbe S, Granstein R D. Mechanisms of ultraviolet radiation carcinogenesis. Chem Immunol 58:291-313, 1994.
3. Grabbe S, Bruvers S, Gallo R L, Knisely T L, Nazareno R, Granstein R D. Tumor antigen presentation by murine epidermal cells. J Immunol 146:3656-61, 1991.
4. Rubel J R, Milford E L, Abdi R. Cutaneous neoplasms in renal transplant recipients. Eur J Dermatol 12:532-5, 2002.
5. Berg D, Otley C C. Skin cancer in organ transplant recipients: Epidemiology, pathogenesis, and management. J Am Acad Dermatol 47:1-17, 2002.
6. Leveque L, Dalac S, Dompmartin A, Louvet S, Euvrard S, Catteau B, Hazan M, Schollhamer M, Aubin F, Dreno B, Daguin P, Chevrant-Breton J, Frances C, Bismuth M J, Tanter Y, Lambert D. Melanoma in organ transplant patients. Ann Dermatol Venereol 127:160-5, 2000.
7. Stingl G, Katz S I, Clement L, Green I, Shevach E M. Immunologic functions of Ia bearing epidermal Langerhans cells. J Immunol 121:2005-13, 1978.
8. Streilen J W, Bergstresser P R. LC: APC of the epidermis. Immunobiology 186:285-300, 1984.
9. Inaba K, Schuler G, Witmer M D, Valinshy J, Atassi B, Steinman R M. Immunologic properties of purified epidermal LC: distinct requirements for the stimulation of unprimed and sensitized T lymphocytes. J Exp Med 164:605-13, 1986.
10. Schuler G, Steinman R M. Murine epidermal Langerhans cells mature into potent immunostimulatory dendritic cells in vitro. J Exp Med 161:526-46, 1985.
11. Witmer-Pack M D, Olivier W, Valinsky J, Schuler G, Steinman R M. Granulocyte/macrophage colony-stimulating factor is essential for the viability and function of cultured murine epidermal Langerhans cells. J Exp Med 166:1484-98, 1987.
12. Peguet-Navarro J, Dalbiez-Gauthier C, Rattis F M, Van Kooten C, Banchereau J, Schmitt D. Functional expression of CD40 antigen on human epidermal Langerhans cells. J Immunol 155:4241-7, 1995.
13. Koyama Y, Marunouchi T. Macrophage colony-stimulating factor (M-CSF) inhibits the decrease in the amount of rRNA and IA beta mRNA in cultured epidermal Langerhans cells of the mouse. J Dermatol 23:73-82, 1996.
14. Borkowski T A, Letterio J J, Farr A G, Udey M C. A role for endogenous transforming growth factor beta 1 in Langerhans cell biology: the skin of transforming growth factor beta 1 null mice is devoid of epidermal Langerhans cells. J Exp Med 184:2417-22, 1996.
15. Beissert S, Hosoi J, Grabbe S, Asahina A, Granstein, R D. IL-10 inhibits tumor antigen presentation by epidermal antigen-presenting cells. J Immunol 154:1280-86, 1995.
16. Grabbe S, Bruvers S, Lindgren A M, Hosoi J, Tan K C, Granstein R D. Tumor antigen presentation by epidermal antigen presenting cells in the mouse: Modulation by granulocyte-macrophage colony stimulating factor, tumor necrosis factor-58 and ultraviolet radiation. J Leuk Biol 52:209-17, 1992.
17. Hosoi J, Egan C L, Lemer E A, Murphy G F, Grabbe S, Granstein R D. Regulation of Langerhans cell function by nerves containing CGRP. Nature 363: 159-63, 1993.
18. Asahina A, Hosoi J, Beissert S, Stratigos A, Granstein R D. inhibition of the induction of delayed-type and contact hypersensitivity by calcitonin gene-related peptide. J Immunol 154:3056-61, 1995.
19. Asahina A, Moro O, Hosoi J, Lerner E A, Xu S, Takashima A, Granstein R D. Specific induction of cyclic AMP in Langerhans cells by calcitonin gene-related peptide: Relevance to functional effects. Proc Natl Acad Sci USA 92:8323-7, 1995.
20. Torii H, Hosoi J, Beissert S, Xu S, Fox F E, Asahina A, Takashima A, Rook A H, Granstein R D. Regulation of cytokine expression in macrophages and the Langerhans cell-like line XS52 by calcitonin gene-related peptide. J Leuk Biol 61:216-223, 1997.
21. Torii H, Yan Z, Hosoi J, Granstein R D. Expression of neurotrophic factors and neuropeptide receptors by Langerhans cells and the Langerhans cell-like line XS52: further suport for a functional relationship between Langerhans cells and epidermal nerves. J Invest Dermatol 109:586-591, 1997.
22. Seiffert K, Hosoi J, Torii H, Ozawa H, Ding W, Campton K, Wagner J A, Granstein R D. Catecholamines inhibit the antigen-presenting capability of epidermal Langerhans cells. J Immunol 168:6128-35, 2002.
23. Di Virgilio F, Chiozzi P, Ferrari D, Falzoni S, Sanz J, Morelli A, Torboli M, Bolognesi G, Baricordi R. Nucleotide receptors: an emerging family of regulatory molecules in blood cells. Blood 97:587-600, 2001.
24. Singh M, O'Hagan D T. Recent advances in vaccine adjuvants. Pharm Res 19:715-28, 2002.
25. Hunter R L. Overview of vaccine adjuvants: present and future. Vaccine 20:S7-S12, 2002.
26. Alving C R. Design and selection of vaccine adjuvants: animal models and human trials. Vaccine 20:S56-S64, 2002.
26a. Ulanov M, Tarkowski A, Hahn-Zoric M, Hanson L A, Moingeon P. The common vaccine adjuvant aluminum hydroxide up-regulates accessory properties of human monocytes via an interleukin 4-dependent mechanism. Infect Immunol 69:1151-1159, 2001.
26b. Shi Y, HogenEsch H, Regnier F E, Hem S L. Detoxification of endotoxin by aluminum hydroxide adjuvant. Vaccine 19:1747-1752, 2001.
27. Panther E, Idzko M, Herouy Y, Rheinen H, Gebicke-Haerter P, Mrowietz U, Dichmann S, Norgauer J. Expression and function of adenosine receptors in human dendritic cells. FASEB J. 15:1963-1970, 2001.
28. Liu Q. H., Bohlen H, Titzer S, Christensen O, Diehl V, Hescheler J, Fleischmann B K. Expression and a role of functionally coupled P2Y receptors in human dendritic cells. FEBS letters 445:402-408, 1999.
29. Berchtold S, Ogilvie A, Bogdan C, Muhl-Zurbes P, Ogilvie A, Schuler G, Steinkasserer A. Human monocyte derived dendritic cells express functional P2X and P2Y receptors as well as ecto-nucleotidases. FEBS letters 458:424-428, 1999.
30. Schnurr M, Then F, Galambos P, Scholz C, Siegmund B, Endres S, Eigler A. Extracellular ATP and TNF-alpha synergize in the activation and maturation of human dendritic cells. J Immunol 22:4704-4709, 2000.
31. Ferrari D, La Sala A, Chiozzi P, Morelli A, Falzoni S, Girolomoni G, Idzko M, Dichmann S, Norgauer J, Di Virgilio F. The P2 purinergic receptors of human dendritic cells: identification and coupling to cytokine release. FASEB J 14:2466-2476, 2000.
32. Wilkin F, Duhant X, Bruyns C, Suarez-Huerta N, Boeynaems J-M, Robaye B. The P2Y₁₁ receptor mediates the ATP-induced maturation of human monocyte-derived dendritic cells. J Immunol 166:7172-77, 2001.
33. la Sala A, Ferrari D, Di Virgilio, Idzko M, Norgauer J, Girolomoni G. Alerting and tuning the immune response by extracellular nucleotides. J Leukoc Biol 73:339-43, 2003.
34. Mizumoto N, Kumamoto T, Robson S, Sevigny J, Matsue H, Enjyoji K, Takashima A. CD39 is the dominant Langerhans cell-associated ecto-NTPDase: modulatory roles in inflammation and immune responsiveness. Nat Med 8:358-365, 2002.
35. Mutini C, Falzoni S, Ferrari D, Chiozzi P, Morelli A, Baricordi O, Collo G, Ricciardi-Castagnoli P, Di Virgilio. Mouse dendritic cells express the P2X₇ purinergic receptor: characterization and possible participation in antigen presentation. J Immunol 163:1958-1965, 1999.
36. Nihei O, de Carvalho A, Savino W, Alves L. Pharmacologic properties of P₂₇Z/P₂X₇ receptor characterized in murine dendritic cells: role on the induction of apoptosis. Blood 96:996-1005, 2000.
37. Coutinho-Silva R, Persechini P, Bisaggio R, Perfettini J-L, De Sa Neto A, Kanellopoulos J, Motta-Ly I, Dautry-Varsat A, Ojcius D. P₂₇Z/P2X₇ receptor-dependent apoptosis of dendritic cells. American Physiological Society 363:1139-1147.
38. Nizet V, Ohtake T, Lauth X, Trowbridge J, Rudisill J, Dorschner R A, Pestonjamasp V, Piraino J, Huttner K, Gallo R L. Innate antimicrobial peptide protects the skin from invasive bacterial infection. Nature 414:454-7, 2001.
39. Jacobson K A, King B F, Bumstock G. Pharmacological characterization of P2 receptors. Newsletter for Cell Signaling and Neuroscience Research 16:3-16, 2000.
40. Martell M, Gomez J, Esteban J I, Sauleda S, Quer J, Cabot B, Esteban R, Guardia J. High-throughput real-time reverse transcription-PCR quantitation of hepatitis C virus RNA. J Clin Microbiol 37:327-332, 1999.
41. Yajima T, Yagihashi A, Kameshima H, Kobayashi D, Furuya D, Hirata K, Watanabe N. Quantitative reverse transcription-PCR asay of the RNA component of human telomerase using the TaqMan fluorogenic detection system. Clin Chem 44:2441-2445, 1998
42. Cho K C, Scarnes W S, Minsk B, Palmieri S, Jackson-Grusby L, Wagner J A. NGF regulates gene expression by several distinct mechanisms. Mol Cell Biol 9:135-143, 1989.
43. Scheibe R J, Wagner J A. Retinoic acid regulates both expression of the nerve growth factor receptor and sensitivity to nerve growth factor. J Biol Chem 267:17611-17616, 1992.
44. Larsen C P, Ritchie S C, Pearson T C, Linsley P S, Lowry R P. Functional expression of the costimulatory molecule B7/BB 1, on murine dendritic cell population. J Exp Med176:1215-1220, 1992.
45. Simon J C, Cruz P D Jr., Tigelaar R E, Sontheimer R D, Bergstresser P R. Adhesion molecules CD11a, CD18 and ICAM-1 on human epidermal Langerhans cells serve a functional role in the activation of alloreactive T cells. J Invest Dermatol 96:148-151, 1991.
46. Ma J, Wang J H, Guo Y J, Sy M S, Bigby M. In vivo treatment with anti-ICAM-1 and anti-LFA-1 antibodies inhibits contact sensitization-induced migration of epidermal Langerhans cells to regional lymph nodes. Cell Immunol 158:389-99, 1994.
47. Bevers E M, Comfurius P, Dekkers D W, Zwaal R F. Lipid translocation across the plasma membrane of mammalian cells. Biochim Biophys Acta 1439:317-30, 1999.
48. Wang B, Zhuang L, Fujisawa H, Shinder G A, Feliciani C, Shivji G M, Suzuki H, Amerio P, Toto P, Sauder D N. Enhanced epidermal Langerhanss cell migration in IL-10 knock-out mice. J Immunol 162:277-83, 1999.
49. Stoitzner P, Holzmann S, McLellan A D, Ivarsson L, Stossel H, Kapp M, Kammerer U, Douillard P, Kampgen E, Koch F, Saeland S, Romani N. Visualization and characterization of migratory Langerhans cells in murine skin and lymph nodes by antibodies against Langerin/CD207. J Invest Dermatol 120:266-74, 2003.
50. Pior J, Vogl T, Sorg C, MacHer E. Free hapten molecules are dispersed by way of the bloodstream during contact sensitization to fluorescein isothiocyanate. J Invest Dermatol 113:888-93, 1999.
51. Kimber I, Cumberbatch M, Dearman R J, Bhushan M, Griffiths CEM. Cytokines and chemokines in the initiation of epidermal Langerhans cell migration. Dr J Dermatol 142:401-12, 2000.
52. Wang B, Kondo S, Shivji G M, Fujisawa H, Mak T W, Sauder D N. Tumour necrosis factor receptor II (p75) signaling is required for the migration of Langerhsn's cells. Immunology 88:284-8,1996.
53. Cumberbatch M, Dearman R J, Kimber I. Langerhans cells require signals from both tumour necrosis factor-alpha and interleukin-1 beta for migration. Immunology 92:388-95, 1997.
54. Gunn M D, Kyuwa S, Tam C, Kakiuchi T, Matsuzawa A, Williams L T, Nakano H. Mice lacking expression of secondary lymphoid organ chemokine have defects in lymphocyte homing and dendritic cell localization. J Exp Med 189:451-60, 1999.
55. Yamazaki S, Yokozeki H, Satoh T, Katayama I, Nishioka K. TNF-alpha, RANTES, and MCP-1 are major chemoattractants of murine Langerhans cells to the regional lymph nodes. Exp Dermatol 7:35-41, 1998.
56. Sato N, Ahuja S K, Quinones M, Kostecki V, Reddick R L, Melby P C, Kuziel W A, Ahuja S S. CC chemokine receptor (CCR)2 is required for langerhans cell migration and localization of T helper cell type 1 (Th1)-inducing dendritic cells. Absence of CCR₂ shifts the Leishmania major-resistant phenotype to a susceptible state dominated by Th2 cytokines, b cell outgrowth, and sustained neutrophilic inflammation. J Exp Med 17;192:205-18, 2000.
57. Larsen C P, Steinman R M, Witmer-Pack M, Hankins D F, Morris P J, Austyn J M. Migration and maturation of Langerhans cells in skin transplants and explants. J Exp Med 172:1483-93, 1990.
58. Saitoh A, Yasaka N, Osada A, Nakamura K, Furoe M, Tamaki K. Migration of Langerhans cells in an in vitro organ culture system: IL-16 and TNF-alpha are partially responsible for migration into the epidermis. J Dermatol Sci 19:166-74, 1999.
59. Ratzinger G, Stoitzner P, Ebner S, Lutz M B, Layton G T, Rainer C, Senior R M, Shipley J M, Fritsch P, Schuler G, Romani N. Matrix metalloproteinases 9 and 2 are necessary for the migration of Langerhans cells and dermal dendritic cells from human and murine skin. J Immunol 168:4361-71, 2002.
60. Stoitzner P, Zanella M, Ortner U, Lukas M, Tagwerker A, Janke K, Lutz M G, Schuler G, Echtenacher B, Ryffel B, Koch F, Romani N. Migration of Langerhans cells and dermal dendritic cells in skin organ cultures: augmentation by TNF-alpha and IL-lbeta. J Leukoc Biol 66:462-70, 1999.
61. Stoitzner R, Ratzinger G, Koch F, Janke K, Scholler T, Kaser A, Tilg H, Cruikshank W W, Fritch P, Romani N. Interleukin-16 supports the migration of Langerhans cells, partly in a CD4 independent way. J Invest Dermatol 116:641-9, 2001.
62. Rupec R, Magerstaedt R, Schirren C G, Sander E, Bieber T. Granulocyte/macropaghe-colony-stimulating factor induces the migration of human epidermal Langerhans cells in vitro. Exp Dermatol 5:115-119, 1996.
63. Yamazaki S, Katayama I, Yokozeki H, Satoh T, Nishioka K. Production of chemotactic factor(s) of Langerhans cells by lymph node cells in contact sensitivity. J Dermatol Sci 11:51-58, 1996.
64. Yamazaki S, Yokozeki H, Satoh T, Katayama I, Nishioka K. TNF□, RANTES, and MCP-1 are major chemoattractants of murine Langerhans cells to the regional lymph nodes. Exp Dermatol 7:35-41, 1998.
65. Leon C, Freund M, Ravanat C, Baurand A, Cazenave J P, Gachet C. Key role of the P2Y(1) receptor in tissue factor-induced thrombin-dependent acute thromboembolism: studies in P2Y(1)-knockout mice and mice treated with a P2Y(1) antagonist. Circulation. 103:718-23, 2001.
66. Zhong Y, Dunn P M, Bardini M, Ford A P, Cockayne D A, Burnstock G. Changes in P2X receptor responses of sensory neurons from P2X3-deficient mice. Eur J Neurosci 14:1784-92, 2001.
67. Robaye B, Ghanem E, Wilkin F, Fokan D, Van Driessche W, Schurmans S, Boeynaems J M, Beauwens R. Loss of nucleotide regulation of epithelial chloride transport in the jejunum of P2Y4-null mice. Mol Pharmacol 63:777-83, 2003.
68. Labasi J M, Petrushova N, Donovan C, McCurdy S, Lira P, Payette M M, Brissette W, Wicks J R, Audoly L, Gabel C A. Absence of the P2X7 receptor alters leukocyte function and attenuates an inflammatory response. J Immunol 168:6436-45, 2002.
69. Larson M K, Chen H, Kahn M L, Taylor A M, Fabre J E, Mortensen R M, Conley P B, Parise L V. Identification of P2Y12-dependent and -independent mechanisms of glycoprotein VI-mediated Rap1 activation in platelets. Blood 101:1409-15, 2003.
70. North R A, Surprenant A. Pharmacology of cloned P2X receptors. Ann Rev Pharmacol Toxicol 40:563-80, 200.
71. von Kugelgen I, Wetter A. Molecular pharmacology of P2Y receptors. Naunyn-Schmiedeberg's Arch Pharmacol 362:310-23, 2000.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. An adjuvant composition comprising a carrier and an effective amount of a purine receptor agonist.

2. The adjuvant composition of claim 1, wherein the purine receptor is on an antigen presenting cell.

3. The adjuvant composition of claim 1, wherein the composition is formulated for intradermal, subcutaneous, intramuscular, intravenous or topical administration.

4. The adjuvant composition of claim 1, wherein the purine receptor agonist is ATP or ATPγS.

5. The adjuvant composition of claim 1, wherein the purine receptor agonist is α, β methylene-ATP (α, β mATP), β, γ methylene-ATP (β-γ mATP), 2-methylthio-ATP (2mSATP), CTP, dATP, UTP, UTPγS, UDP, α, β methylene-adenosine 5′-triphosphate (α, β mATP), D-β,γ methylene-adenosine 5′-triphosphate (D-β, γ mATP), 2-methylthio-adenosine 5′-triphosphate (2mSATP), 2-methylthioadenosine 5′-triphosphate, 2′,3′-O-(4-benzoylbenzoyl)adenosine 5′-triphosphate, hexythioadenosine 5-monophosphate, 2-[2-(4-aminophenyl)ethylthio]adenosine 5′-triphosphate, diadenosine tetraphosphate, diadenosine pentaphosphate, adenosine 5′-O-(2-thiodiphosphate), 2-methylthioadenosine 5′-diphosphate, 2-propylthio-D-β, γ-dichloromethylene-ATP or uridine 5′-O-(2-thiodiphosphate).

6. The adjuvant composition of claim 1, wherein the purine receptor agonist is a compound of formula I or II: 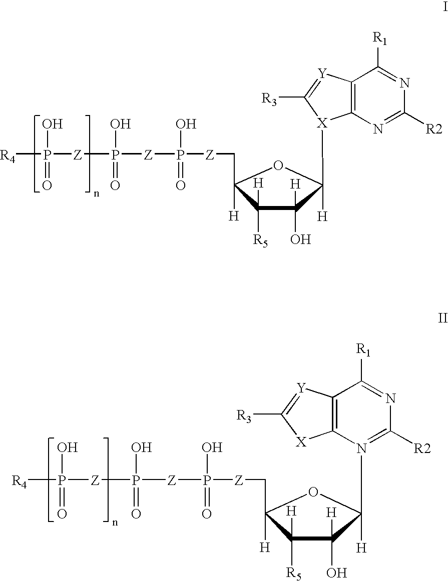
wherein:
R₁ and R₂, independently, are halogen or —R₆—(R₇)ₚ—R₈;
R₃ is H, halogen or —R₆—(R₇)ₚ—R₈, wherein p is 0 or 1;
R₄ is OH or SH;
R₅ is OH or acetamido;
R₆ is NH or S;
R₇ is H or alkylene having from 1 to 10 carbon atoms;
R₈ is H, NH₂, CN, cycloalkyl having 3 to about 10 carbon atoms, or aryl having 3 to about 20 carbon atoms;
X and Y are independently N or CH;
Z is O, S or CH₂; and
n is 0 or 1.

7. The composition of claim 1, wherein the effective amount of a purine receptor agonist is a pharmaceutically effective amount of the purine receptor agonist.

8. A composition comprising an antigen and an effective amount of a purine receptor agonist.

9. A composition for treating an undesirable immune response comprising a carrier and an effective amount of a purine receptor antagonist.

10. The composition of claim 9, wherein the purine receptor antagonist can inhibit a purine receptor.

11. The composition of claim 9, wherein the purine receptor antagonist is oxidized ATP, pyridoxal-5′-phosphate-6-(2′-naphthylazo-6′-nitro-4′,8′-disulfonate), pyridoxal-5′-phosphate-6-azophenyl-2′,5′-disulfonic acid, pyridoxal-5′-phosphate-6-azophenyl-2′,4′-disulfonic acid, pyridoxal-5′-phosphate-6-azophenyl-4′-carboxylate, diinosine pentaphosphate, 8,8′-(carbonylbis(imino-3,1-phenylene carbonylimino)bis(1,3,5-naphthalenetrisulfonic acid), 8,8′-(carbonylbis(imino-4,1-phenylene carbonylimino-4,1-phenylene carbonylimino)bis( 1,3,5-naphthalenetrisulfonic acid), Suramin, 2′,3′-O-(2,4,6-trinitrophenyl) adenosine triphosphate, reactive blue 2, brilliant blue G, 1-[N, O-bis(5-isoquinolonesulfonyl)-N-methyl-L-tyrosyl]-4-phenylpiperazine, hexamethylene amioride, oxidized ATP, adenosine 3′-phosphate 5′-phosphosulfate, 2′-deoxy-N⁶-methyladenosine-3′,5′-bisphosphate, (N)-methanocarba-N⁶-methyl-2-chloro-2′-deoxyadenosine-3′,5′-bisphosphate, 2-propylthio-D-β, γ-dichloromethylene-ATP, N⁶-[2-(methylthio)-ethyl]-2-(3,3,3-trifluoropropyl)thio-5′-adenylic acid, or N¹-(6-ethoxy-1,3-benzothiazol-2-yl-2(7-ethoxy-4-hydroxy-2,2-dioxo-2H-2-⁶benzo[4,5][1,3] thiazolo[2,3-c][1,2,4]thiadiazin-3-yl)-2oxo-1-ethanesulfonamide, 2-methylthioadenosine 5′-monophosphate.

12. The composition of claim 9, wherein the undesirable immune response is an autoimmune disease, or a negative reaction against a therapeutic agent.

13. The composition of claim 9, wherein the autoimmune disease is diabetes mellitus, arthritis, rheumatoid arthritis, juvenile rheumatoid arthritis, osteoarthritis, psoriatic arthritis, multiple sclerosis, myasthenia gravis, systemic lupus erythematosis, autoimmune thyroiditis, dermatitis, atopic dermatitis, eczematous dermatitis, psoriasis, Sjogren's Syndrome, keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves ophthalmopathy, sarcoidosis, primary biliary cirrhosis, uveitis posterior, or interstitial lung fibrosis.

14. The composition of claim 9, wherein the composition is formulated for intradermal, subcutaneous, intramuscular, intravenous or topical administration.

15. The composition of claim 9, wherein the purine receptor antagonist is formulated for administration at a site of inflammation.

16. A method for stimulating an immune response in an animal comprising administering to the animal an antigen and an adjuvant composition comprising an effective amount of a purine receptor agonist.

17. The method of claim 16, wherein the purine receptor agonist can stimulate a purine receptor.

18. The method of claim 16, wherein the purine receptor is on an antigen presenting cell.

19. The method of claim 16, wherein the purine receptor agonist is formulated for intradermal, subcutaneous, intramuscular, intravenous or topical administration.

20. The method of claim 16, wherein the purine receptor agonist is ATP or ATPγS.

21. The method of claim 16, wherein the purine receptor agonist is α, β methylene-ATP (α, β mATP), β, γ methylene-ATP (β, γ mATP), 2-methylthio-ATP (2mSATP), CTP, dATP, UTP, UTPγS, UDP, α, β methylene-adenosine 5′-triphosphate (α, β mATP), D-β, γ methylene-adenosine 5′-triphosphate (D-β, γ mATP), 2-methylthio-adenosine 5′-triphosphate (2mSATP), 2-methylthioadenosine 5′-triphosphate, 2′,3′-O-(4-benzoylbenzoyl)adenosine 5′-triphosphate, hexythioadenosine 5-monophosphate, 2-[2-(4-aminophenyl)ethylthio]adenosine 5′-triphosphate, diadenosine tetraphosphate, diadenosine pentaphosphate, adenosine 5′-O-(2-thiodiphosphate), 2-methylthioadenosine 5′-diphosphate, 2-propylthio-D-β, γ-dichloromethylene-ATP or uridine 5′-O-(2-thiodiphosphate).

22. The method of claim 16, wherein the purine receptor agonist is a compound of formula I or II: 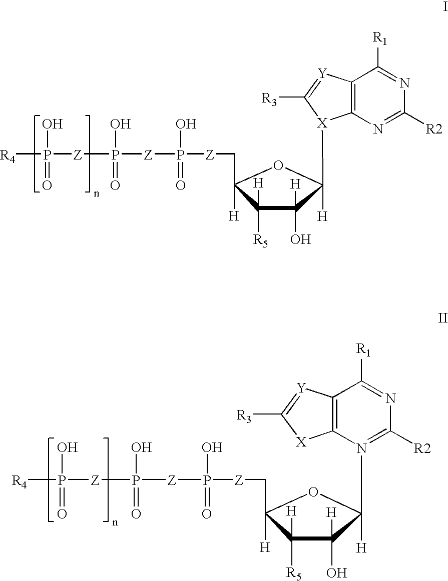
wherein:
R₁ and R₂, independently, are halogen or —R₆—(R₇)ₚ—R₈;
R₃ is H, halogen or —R₆—(R₇)ₚ—R₈, wherein p is 0 or 1;
R₄ is OH or SH,
R₅ is OH or acetamido;
R₆ is NH or S;
R₇ is alkylene having from 1 to 10 carbon atoms;
R₈ is H, NH₂, CN, cycloalkyl having 3 to about 10 carbon atoms, or aryl having 3 to about 20 carbon atoms;
X and Y are independently N or CH;
Z is O, S or CH₂; and
n is 0 or 1.

23. A method of stimulating an immune response to an immunogen comprising topically administering a composition comprising the immunogen and an effective amount of ATP or ATPγS.

24. A method for treating or preventing an undesirable immune response in an animal comprising administering to the animal an effective amount of a purine receptor antagonist.

25. The method of claim 24, wherein the purine receptor antagonist can inhibit a purine receptor.

26. The method of claim 24, wherein the purine receptor antagonist is oxidized ATP, pyridoxal-5′-phosphate-6-(2′-naphthylazo-6′-nitro-4′,8′-disulfonate), pyridoxal-5′-phosphate-6-azophenyl-2′,5′-disulfonic acid, pyridoxal-5′-phosphate-6-azophenyl-2′,4′-disulfonic acid, pyridoxal-5′-phosphate-6-azophenyl-4′-carboxylate, diinosine pentaphosphate, 8,8′-(carbonylbis(imino-3,1-phenylene carbonylimino)bis(1,3,5-naphthalenetrisulfonic acid), 8,8′-(carbonylbis(imino-4,1-phenylene carbonylimino-4,1-phenylene carbonylimino)bis( 1,3,5-naphthalenetrisulfonic acid), Suramin, 2′,3′-O-(2,4,6-trinitrophenyl) adenosine triphosphate, reactive blue 2, brilliant blue G, 1-[N, O-bis(5-isoquinolonesulfonyl)-N-methyl-L-tyrosyl]-4-phenylpiperazine, hexamethylene amioride, oxidized ATP, adenosine 3′-phosphate 5′-phosphosulfate, 2′-deoxy-N⁶-methyladenosine-3′,5′-bisphosphate, (N)-methanocarba-N⁶-methyl-2-chloro-2′-deoxyadenosine-3′,5′-bisphosphate, 2-propylthio-D-β, γ-dichloromethylene-ATP, N⁶-[2-(methylthio)-ethyl]-2-(3,3,3-trifluoropropyl)thio-5′-adenylic acid, or N¹-(6-ethoxy-1,3-benzothiazol-2-yl-2(7-ethoxy-4-hydroxy-2,2-dioxo-2H-2-⁶benzo[4,5][1,3] thiazolo[2,3-c][1,2,4]thiadiazin-3-yl)-2oxo-1-ethanesulfonamide, 2-methylthioadenosine 5′-monophosphate.

27. The method of claim 24, wherein the undesirable immune response is an autoimmune disease, or a negative reaction against a therapeutic agent.

28. The method of claim 27, wherein the autoimmune disease is diabetes mellitus, arthritis, rheumatoid arthritis, juvenile rheumatoid arthritis, osteoarthritis, psoriatic arthritis, multiple sclerosis, myasthenia gravis, systemic lupus erythematosis, autoimmune thyroiditis, dermatitis, atopic dermatitis, eczematous dermatitis, psoriasis, Sjogren's Syndrome, keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves ophthalmopathy, sarcoidosis, primary biliary cirrhosis, uveitis posterior, or interstitial lung fibrosis.

29. The method of claim 24, wherein the purine receptor antagonist is formulated for intradermal, subcutaneous, intramuscular, intravenous or topical administration.

30. The method of claim 24, wherein the purine receptor antagonist is formulated for administration at a site of inflammation.

31. The method of claim 24, wherein the purine receptor antagonist is administered as a transdermal patch.
